(19) United States
(12) Patent Application Publication
(10) Pub. No.: US 2008/0269202 A1
(43) Pub. Date: Oct. 30, 2008

(54) Novel 2,3-benzodiazepine derivatives and their use as antipsychotic agents

(75) Inventors: Emese Csuzdi, Budapest (HU); Sandor Solyom, Szigetmonostor (HU); Pal Berzsenyi, Budapest (HU); Ferenc Andrasi, Budapest (HU); Istvan Sziraki, Budapest (HU); Katalin Horvath, Solymar (HU); Zoltan Nagy, Isaszeg (HU)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Teva Pharmaceutical Industries Ltd., Petach Tikva (IL)

(21) Appl. No.: 12/080,418

(22) Filed: Apr. 2, 2008

Related U.S. Application Data
(60) Provisional application No. 60/921,532, filed on Apr. 2, 2007.
(60) Provisional application No. 60/936,631, filed on Jun. 20, 2007.

(51) Int. Cl. A61K 31/551; C07D 243/00; A61P 25/00

(52) U.S. Cl. 514/221; 540/567

(57) ABSTRACT

Disclosed are novel 2,3-benzodiazepine derivatives and methods of making the same.

### CROSS-REFERENCE TO RELATED APPLICATIONS

This application claims the benefit of the filing date of U.S. Provisional Patent Application No. 60/921,532 filed Apr. 2, 2007; and U.S. Provisional Patent Application No. 60/936,631 filed Jun. 20, 2007, the disclosures of which are hereby incorporated herein by reference.

### BACKGROUND OF THE INVENTION

Conventional antipsychotics (for example, chlorpromazine or haloperidol) exert their effect by the antagonism of the dopamine-D₂ receptor. However, about 30% of psychotic patients are refractory to the current antidopamine therapy. Thus, atypical antipsychotics having better side-effect profiles, which differ in mechanism from the classical ones, have gained special importance.

Within the family of 2,3-benzodiazepines, several groups of compounds have been reported to have high therapeutic value in different therapeutic areas related to the central nervous system (CNS) (for reviews see, for example, Horváth, E. J., Horváth, K. et al, Progress in Neurobiology 2000, 60, 309 and Sólyom, S., Tarnawa, I. Curr. Pharm. Des. 2002, 8, 9-13).

Among the 5H-[2,3]benzodiazepines, the compound 7,8-dimethoxy-1-(3,4-dimethoxyphenyl)-5-ethyl-4-methyl-5H-[2,3]benzodiazepine (Grandaxin) is a non-sedative anxiolytic. Hungarian Patent No. 179 018 describes the anxiolytic compound 7,8-dimethoxy-1-(3-chlorophenyl)-5H-[2,3]benzodiazepine (Girisopam) as a follow-up compound. Another related compound, 1-(4-aminophenyl)-7,8-dimethoxy-5H-[2,3]benzodiazepine (Nerisopam), is disclosed in Hungarian Patent No. 191 698 as having some definite antipsychotic character in addition to having an anxiolytic effect.

Hungarian Patent Nos. 221 508, 224 435, and 224 438 disclose 5H-[2,3]benzodiazepine derivatives bearing substituted styryl groups in position 1 and alkoxy or methylenedioxy substituents in positions 7,8. These compounds are disclosed to have different CNS activities, for example, anxiolytic, antiaggresive and antipsychotic effects.

A quite different biological activity profile was shown when the 7,8-dimethoxy substituents of Nerisopam, were replaced with a methylenedioxy group. The compound 5-(4-aminophenyl)-8-methyl-9H-1,3-dioxolo[4,5-h][2,3]benzodiazepine had an anticonvulsive effect and was found to be a non-competitive AMPA antagonist belonging to the family of glutamate antagonists (Tarnawa et al. Eur. J. Pharmacol, 1989, 167, 193; Smith, S. E., Meldrum, B. S. Eur. J. Pharmacol 1990, 187, 131; U.S. Pat. No. 4,614,740).

Compounds with similar AMPA antagonist activity were found in 2,3-benzodiazepine systems containing a dioxolane ring at the 7,8 position or in systems containing halogen atoms at positions 7 and/or 8. Moreover, substitution of a methoxy group at either position 7 or 8 of the 4,5-dihydro-3H-[2,3]benzodiazepine or 3H-[2,3]benzodiazepine systems and further bearing an acyl substituent in position 3, also showed similar AMPA antagonist activity. Such compounds are described, for example, in Hungarian Patent Nos. 191 698; 191 702; 206 719; 219 777; in the U.S. Pat. Nos. 5,459,137; 5,536,832; in British Patent No. 2 311 779, as well as WO 96/04 283, WO 97/28 135 (U.S. Pat. No. 6,200,970), WO 99/07 707, WO 99/07 708, WO 01/04 122 and WO 05/01 2265.

Other 2,3-benzodiazepine derivatives with AMPA antagonist activities, which bear a 5- or 6-membered heterocyclic substituent at N-3 and having a methylenedioxy, halogen, or methoxy substituent at positions 7 or 8 have been disclosed. See, for example, U.S. Pat. Nos. 5,795,886 and 6,858,605.

U.S. Pat. No. 6,887,867 (hereinafter the “'867 Patent”) (PCT Application No. WO-01/98280) discloses, 2,3-benzodiazepine derivatives as exerting non-NMDA excitatory aminoacid (AMPA) antagonist activity. However, neither the identification data, the physical characteristics, or the process of preparation of the claimed 2,3-benzodiazepines bearing two C₁-C₃ alkoxy substituents in positions 7,8 is disclosed in the '867 patent.

### SUMMARY OF THE INVENTION

The invention relates to new 2,3-benzodiazepine derivatives of formula (I), isomers and acid addition salts thereof,

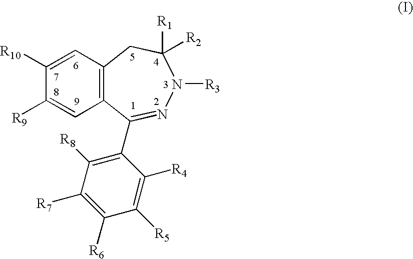

wherein R¹ is methyl and R² is hydrogen;
R³ represents one of the following:
(a) a 5 or 6 membered heterocyclic ring which is either aromatic, saturated or partially saturated, said heterocyclic ring containing 1, 2, or 3 heteroatoms selected from the group consisting of O, S, or N, said heterocyclic ring optionally substituted by a C₁-C₃ alkyl group, a C₂-C₃ alkenyl group or an oxo group,

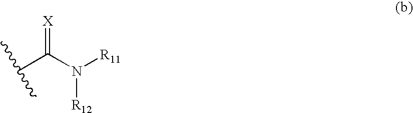

wherein X is O or S;
R¹¹ is hydrogen, C₁-C₄ alkyl or cycloalkyl or phenyl, and
R¹² is C₁-C₄ alkyl, cycloalkyl, phenyl, or C₁-C₃ alkoxy, or
R¹¹ and R₁₂ together with the nitrogen atom to which they are attached form an imidazolyl or a morpholinyl group, or

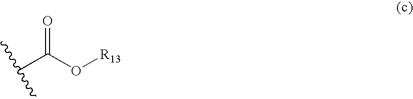

wherein R¹³ is C₁-C₄ alkyl or phenyl;
R⁴, R⁵, R⁶, R⁷, and R⁸ are each independently H, halogen, C₁-C₃ alkyl, nitro, NR¹⁵R¹⁶ wherein R¹⁵ and R¹⁶ can each independently be H, C₁-C₃ alkyl, C₂-C₅ acyl, C₂-C₅ alkoxycarbonyl, aminocarbonyl, or C₁-C₅ alkylaminocarbonyl; and
R⁹ and R¹⁰ are each independently C₁-C₃ alkoxy.

The invention also discloses pharmaceutical compositions comprising a compound of formula (I) as the active ingredient, or a stereoisomer or a pharmaceutically acceptable salt thereof. The composition may further comprise a pharmaceutically acceptable carrier, e.g., solvents, diluents, and fillers.

The compounds of formula (I) are suitable for treating psychotic disorders, including schizophrenia, schizophreniform disorder, schizoaffective disorder, delusional disorder, brief psychotic disorder, shared psychotic disorder, psychotic disorder due to a general medical condition, substance-induced psychotic disorder, psychotic disorder not otherwise specified, bipolar disorder and mood disorders with psychotic symptoms.

Accordingly, a further aspect of the present invention is directed to methods of treating psychotic disorders comprising administering to a subject in need thereof a therapeutically effective amount of a compound of formula (I) or a stereoisomer or a pharmaceutically acceptable salt thereof.

Applicants have surprisingly found that 2,3-benzodiazepine derivatives bearing a methoxy substituent at each of positions 7 and 8, in addition to having a carbamoyl group at N-3 (2,3-benzodiazepine numbering), do not exhibit AMPA antagonism. On the other hand, applicants have also discovered that these compounds show antipsychotic activity.

### BRIEF DESCRIPTION OF THE DRAWINGS

FIG. 1 shows, comparatively, the effect of clozapine, compound 121 and compound 183 on PCP-induced disruption of pre-pulse intensity. Data represent mean ±SEM.

FIG. 2 shows, comparatively, the effect of compounds 121 (“CMP A”) and 183 (“CMP B”) on locomotor behavior in mice. Data represent mean ±SEM.

### DETAILED DESCRIPTION

The invention provides new 2,3-benzodiazepine derivatives of formula (I), the isomers as well as the acid addition salts thereof,

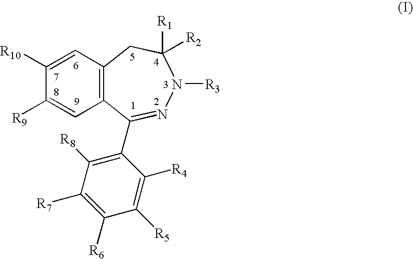

wherein R¹ is methyl and R² is hydrogen,
R³ represents one of the following:
(a) a 5 or 6 membered heterocyclic ring which is either aromatic, saturated or partially saturated, said heterocyclic ring containing 1, 2, or 3 heteroatoms selected from the group consisting of O, S, or N, said heterocyclic ring optionally substituted by a C₁-C₃ alkyl group, a C₂-C₃ alkenyl group or an oxo group;

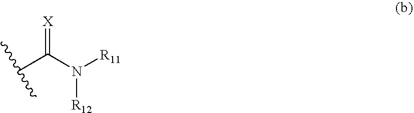

wherein X represents O or S;
R¹¹ is hydrogen, C₁-C₄ alkyl, cycloalkyl or phenyl, and
R¹² is C₁-C₄ alkyl, cycloalkyl, phenyl, or C₁-C₃ alkoxy, or
R¹¹ and R₁₂ together with the nitrogen atom to which they are attached form an imidazolyl or a morpholinyl group; or

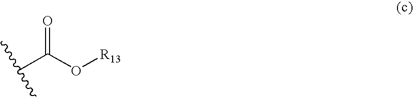

wherein R¹³ represents C₁-C₄ alkyl or phenyl;
R⁴, R⁵, R⁶, R⁷, and R⁸ are each independently H, halogen, C₁-C₃ alkyl, nitro, NR¹⁵R¹⁶ wherein R¹⁵ and R¹⁶ can each independently be H, C₁-C₃ alkyl, C₂-C₅ acyl, C₂-C₅ alkoxycarbonyl, aminocarbonyl, or C₁-C₅ alkylaminocarbonyl; and
R⁹ and R¹⁰ are each independently C₁-C₃ alkoxy.

The term alkyl group encompasses both straight and branched chain alkyl groups. The meaning of alkenyl group can be vinyl, 1-propenyl or 2-propenyl group. Halogen atoms can be fluorine, chlorine, bromine or iodine atom. The amino group can be unsubstituted or substituted with one or two alkyl groups as well as acylated with aliphatic or aromatic carboxylic acids or any kind of carbonic acid esters.

The heterocyclic substituent of the 2,3-benzodiazepine ring as R₃ can be, among others, thiazole, thiazoline, 4-thiazolinone, oxazole, oxazoline, 1,3,4-thiadiazole, 1,3,4-oxadiazole, 1,2,4-thiadiazolin-3-one, 1,2,4-oxadiazole, 4H-1,2,4-oxadiazol-5-one, 1,4,2-oxathiazole, 1,3,4-triazole, pyridine and 5,6-dihydro-4H-[1,3,4]thiadiazin-5-one.

In case of compounds of formula (I), the term “isomers” or “stereoisomers” includes both R and S enantiomers, as well as E and Z isomers, if applicable. Furthermore, “isomers” shall include diasteromers, tautomers and mixtures thereof, for example racemates.

Salts of the compounds of formula (I) relate to physiologically and/or pharmaceutically acceptable salts formed with inorganic or organic acids. Suitable inorganic acids can be, for example, hydrochloric acid, hydrobromic acid, phosphoric acid or sulfuric acid. Suitable organic acids can be, for example, formic acid, acetic acid, maleic acid, fumaric acid, succinic acid, lactic acid, tartaric acid, citric acid or methanesulfonic acid.

In some embodiments, R⁹ and R¹⁰ are both methoxy and R³ is a 5- or 6-membered heterocyclic ring which is either aromatic, saturated or partially saturated, wherein the heterocyclic ring contains 1, 2, or 3 heteroatoms selected from the group consisting of O, S, or N, and wherein said heterocyclic ring is optionally substituted by a C₁-C₃ alkyl group, a C₂-C₃ alkenyl group or an oxo group.

In other embodiments, R⁹ and R¹⁰ are both methoxy and R³ a substituted or unsubstituted thiazole, thiazoline, 4-thiazolinone, oxazole, oxazoline, 1,3,4-thiadiazole, 1,3,4-oxadiazole, 1,2,4-thiadiazolin-3-one, 1,2,4-oxadiazole, 4H-1,2,4-oxadiazol-5-one, 1,4,2-oxathiazole, 1,3,4-triazole, pyridine or 5,6-dihydro-4H-[1,3,4]thiadiazin-5-one.

In yet other embodiments, R⁹ and R¹⁰ are both methoxy and R³ is

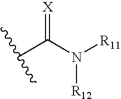

wherein X is O or S;
R¹¹ is hydrogen, C₁-C₄ alkyl or cycloalkyl or phenyl, and
R¹² is C₁-C₄ alkyl, cycloalkyl, phenyl, or C₁-C₃ alkoxy, or
R¹¹ and R¹² together with the nitrogen atom to which they are attached form an imidazolyl or a morpholinyl group.

In yet other embodiments, R⁹ and R¹⁰ are both methoxy and R³ is

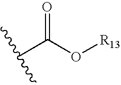

wherein R¹³ is C₁-C₄ alkyl or phenyl.

In yet further embodiments, R³ is 1,3-thiazol-2-yl, R⁹ and R¹⁰ are each methoxy, and the stereochemistry of the carbon in the 4-position is in the R-conformation.

One or more representative compounds of formula (I) of the invention include the following: [R]-1-(4-aminophenyl)-7,8-dimethoxy-4-methyl-3-(1,3-thiazol-2-yl)-4,5-dihydro-3H-[2,3]benzodiazepine; [R]-1-(4-N-acetylaminophenyl)-7,8-dimethoxy-4-methyl-3-(1,3-thiazol-2-yl)-4,5-dihydro-3H-[2,3]benzodiazepine; [R]-1-(4-amino-3-methylphenyl)-7,8-dimethoxy-4-methyl-3-(1,2,4-oxadiazol-3-yl)-4,5-dihydro-3H-[2,3]benzodiazepine; [R]-1-(4-amino-3-methylphenyl)-7,8-dimethoxy-4-methyl-3-(1,3,4-thiadiazol-2-yl)-4,5-dihydro-3H-[2,3]benzodiazepine; [R]-1-(4-aminophenyl)-7,8-dimethoxy-4-methyl-3-methylcarbamoyl-4,5-dihydro-3H-[2,3]benzodiazepine; [R]-1-(4-aminophenyl)-7,8-dimethoxy-4-methyl-3-methylcarbamoyl-4,5-dihydro-3H-[2,3]benzodiazepine; [R]-1-(4-amino-3-methylphenyl)-7,8-dimethoxy-4-methyl-3-methylcarbamoyl-4,5-dihydro-3H-[2,3]benzodiazepine; and the acid addition salts thereof.

The compounds of formula (I) can be prepared in the following manner from a compound of formula (II)

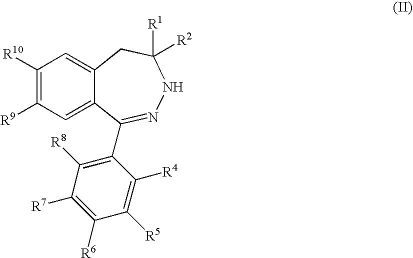

wherein the meaning of R¹, R², R⁴, R⁵, R⁶, R⁷, R⁸, R⁹ and R¹⁰ is as defined above and the heterocycle, corresponding to R³ of formula (I), is linked by known methods.

Alternatively, a compound of general formula (II) can be reacted with an active derivative of a carbamoic acid of formula (III)

R¹¹R¹²N—CX-Z (III)

wherein the meanings of R¹¹ and R¹² are as defined above and Z is halogen atom or a leaving group. Alternatively, a compound of general formula (II) is reacted with an isocyanate or an isothiocyanate of formula (IV)

R¹¹—N═C═X (IV)

wherein the meanings of R¹¹ and X are as defined above.

Alternatively, a compound of formula (II) is reacted with an activated carbonic acid derivative of formula (V)

R¹³—O—CO-Z (V)

wherein the meaning of R¹³ is as defined above and Z is halogen atom or a leaving group.

Alternatively, a compound of formula (I) wherein the meanings of R¹, R², R⁴, R⁵, R⁶, R⁷, R⁸, R⁹ and R¹⁰ is as defined above and R³ is such a group R¹³O—CO— wherein R¹³ is a phenyl group, or the meaning of R³ is such a group R¹¹R¹²N—CO— wherein R¹¹ and R¹² together mean an imidazolyl group, can be formed by reacting a compound of formula (II) with an amine of general formula (VI)

R¹¹R¹²NH (VI)

wherein the meanings of R¹¹ and R¹² are as defined above, other than an imidazolyl group.

To synthesize the compounds of the invention, a compound of formula (VII) or an isochromenilium salt of formula (VIIa) which is formed from a compound of formula (VII)

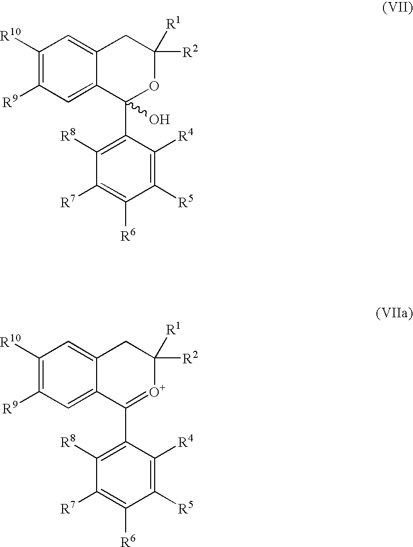

wherein the meaning of R¹, R², R⁴, R⁵, R⁶, R⁷, R⁸, R⁹ and R¹⁰ is as defined above, is reacted with a compound of formula (VIII) or (IX)

R³—NH—NH₂ (VIII)

R¹⁴—NH—NH₂ (IX)

wherein the meaning of R³ is as defined above and the meaning of R¹⁴ is C₂-C₈ alkoxycarbonyl or aryl alkoxycarbonyl group, to obtain the compounds of formulas (X) or (XI).

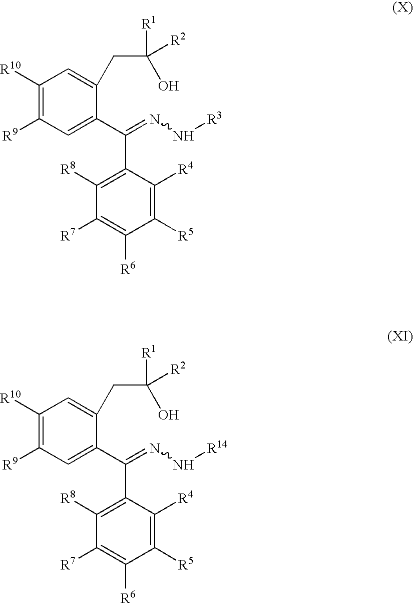

The hydroxyl group of the compounds of formulas (X) or (XI) is transformed into a sulfonate ester, and the latter intermediate is subjected to ring-closure resulting in compounds of formulas (I) or (XII)

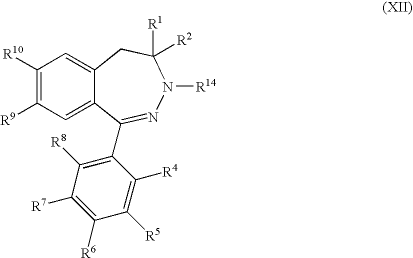

by reaction with a strong base.

In the compounds of formula (XII), the R¹⁴ group can then cleaved to produce a compound of formula (II), which is converted into compounds of formula (I) according to methods described above. Then, if desired, the nitro group of a compound of formula (I) is reduced. Alternatively, the amino group is acylated, alkylated or, after diazotation, is exchanged with a halogen or hydrogen atom. The halogen atom of the resulting acyl group is exchanged with an amino group or the resulting a carbonyl group is thionated to give a thiocarbonyl derivative.

The compounds of formula (II) and (XII) are chiral compounds and therefore formulas (II) and (XII) refer to either of the individual enantiomers or mixtures thereof.

The hemiketal type compounds of formula (VII) as well as the hydrazone derivatives of formula (X) and (XI) represent different stereoisomers and they refer to all possible individual stereoisomers and mixtures thereof.

The R¹⁴ group can be a C₂-C₈ alkoxycarbonyl or a benzyloxycarbonyl group. The cleavage of the R¹⁴ group can be achieved either by acidic or hydrogenolytic methods.

A leaving group, during the above transformations, can be without limitation a substituted or unsubstituted benzenesulfonate group, phenoxy group or an alkanesulfonate group, especially methanesulfonate group or an imidazolyl group.

A racemic starting material of formula (II) and related racemic derivatives of 7,8-dimethoxy (or dialkoxy)-4-methyl-1-(substituted) phenyl-4,5-dihydro-3H-[2,3]benzodiazepines are known in the scientific literature and are described in Belgian Patent No. 892395 (U.S. Pat. No. 4,423,044). See also HU 186 760.

Optically active compounds of formula (II) can be synthesized from an optically active, substituted phenyl-isopropanol according to Anderson et al. (J. Am. Chem. Soc. 1995, 117, 12358). Thus, e.g. starting from (S)-3,4-dimethoxyphenyl-isopropanol (Erdélyi, B. et al. Tetrahedron: Asymmetry 2006, 17, 268), a hemiketal of formula (VII) can be synthesized according to methods described by Anderson et al., supra., and reacting this compound instead of acethydrazid with an alkoxycarbonyl-hydrazid, such as tert-butyl carbazate, containing an easily removable tert-butoxycarbonyl group, the hydrazon of formula (XI) can be obtained. The hydrazon can be transformed, e.g. with methanesulfonyl chloride in the presence of triethylamine, into a mesyloxy derivative. This compound is then treated with base, for example sodium hydroxide in alcoholic solution, to yield the benzodiazepine derivative of formula (XII) in a ring closure reaction. The R¹⁴ substituent of the N-3 atom (2,3-benzodiazepine numbering) is then cleaved, e.g. by hydrolysis or another suitable method, to yield the desired compound of formula (II). The cleavage of the tert-butoxycarbonyl group may be carried out with trifluoroacetic acid, hydrochloric acid, or zinc bromide in dichloromethane. Thus, regarding the inversion of configuration during the synthetic sequence, e.g. from a substituted (S)-phenyl-isopropanol, a compound of formula (II) with (R) configuration can be formed. Whereas, if e.g. 3,4-dimethoxyphenyl-acetone is reduced microbiologically with the strain Debaryomyces carsonii IDR 513, similarly to the method described by Anderson et al., supra., (R)-3,4-dimethoxyphenyl-isopropanol is formed, which after similar transformations as described before, gives rise to the formation of benzodiazepines of formula (II) with (S) configuration.

In those molecules of formula (I), wherein the R³ substituent is a heterocycle, the heterocyclic moiety can be built up starting from compounds of formula (II) according to methods known in the art relating to heterocyclic chemistry.

Some of the compounds of formula (I), wherein R³ is a sulfur containing heterocycle, can be synthesized e.g. from 4,5-dihydro-3H-[2,3]benzodiazepine derivatives substituted with a thiocarbamoyl group at position 3 of the benzodiazepine ring. These thiocarbamoyl compounds can be obtained from 4,5-dihydro-3H-[2,3]benzodiazepine derivatives of formula (II), for example with potassium thiocyanate in acetic acid medium. The thus obtained 4,5-dihydro-3-thiocarbamoyl-3H-[2,3]benzodiazepines, when reacted with α-halo-ketones or α-halo-aldehyde acetals give 2-thiazolyl substituted 2,3-benzodiazepine derivatives. In an analogous reaction, if 2-halo-carboxylic acid esters are used instead of the α-halo oxo-compound, the appropriate compounds, containing a 3-thiazolinone ring, are formed. Similarly, if the 3-thiocarbamoyl-2,3-benzodiazepine intermediate is reacted with 1,2-dibromoethane or β-bromoethylamine, benzodiazepines substituted with a 2-thiazoline ring are formed.

The compounds of formula (I) containing a 1,3,4-thiadiazole group as the R³ substituent can be synthesized for example as follows: First the 3,5-dihydro-3H-[2,3]benzodiazepine of formula (II) is reacted with thiophosgene in the presence of triethylamine to give the corresponding thiocarboxylic acid chloride and the latter is then reacted with hydrazine to yield the thiocarboxylic acid hydrazide derivatives. Latter 2,3-benzodiazepine-3-carbothiohydrazide derivatives are reacted with an acid anhydride or chloride to attain carbothio-N-acylhydrazides. The ring closure of the carbothio-N-acylhydrazides is promoted by further acid treatment to yield the [1,3,4]thiadiazol-2-yl-2,3-benzodiazepines. In another synthetic procedure of the latter compounds the above mentioned intermediate thiocarboxylic acid chloride is first reacted with an acid hydrazide and the resulting carbothiohydrazide derivative containing an acyl group on the terminal N-atom is treated with acid to result in the cyclic product.

If the above described intermediate N-acyl-thiocarboxylic acid hydrazide derivatives are treated with a sulfur binding agent, for example mercury (II) acetate, then benzodiazepines of formula (I) can be obtained, wherein the R³ substituent is an [1,3,4]oxadiazole ring.

Compounds of formula (I), wherein R³ is a six membered 2-(5-oxo-5,6-dihydro-4H-[1,3,4]thiadiazin-2-yl) group, can be prepared by reacting the aforementioned 4,5-dihydro-[2,3]benzodiazepine-3-carbothiohydrazide intermediate with bromoacetic acid ester.

Reacting a 4,5-dihydro-[2,3]benzodiazepine-3-thiocarboxylic acid chloride with hydroxylamine the corresponding thiohydroxamic acid can be obtained and the latter can be transformed into a heterocyclic compound by reacting with a bifunctional alkylating reagent. For example compounds of formula (I), wherein R³ is a [1,4,2]oxathiazol-3-yl group can be obtained when an above mentioned 4,5-dihydro-3H-2,3-benzodiazepine-3-thiohydroxamic acid is reacted with methylene iodide.

The compounds of formula (I) with (3-oxo-2,3-dihydro-[1,2,4]thiadiazol-5-yl) group as the R³ substituent can be prepared, for example, by reacting the unsubstituted compounds of formula (II) with phenoxycarbonyl isothiocyanate, then the resulting 3-(phenoxycarbonyl-thiocarbamoyl)-benzodiazepine is transformed into 3-(N′-alkyl-carbamoyl)-thiocarbamoyl-benzodiazepine with primary amines and the latter is reacted with bromine to accomplish the ring closure between the sulfur and the nitrogen atoms.

The compounds of formula (I) with a (4,5-dihydro-oxazol-2-yl) group as an R³ substituent can be synthesized by reacting the compound of formula (II) with chloroethyl isocyanate to give the urea intermediate, which is then heated in the presence of sodium iodide and potassium carbonate in dimethylformamide to accomplish ring closure.

Compounds of formula (I) containing an unsubstituted or 5-alkyl substituted ([1,2,4]oxadiazole-3-yl) group as R³ substituent can be synthesized for example from 3-cyano-4,5-dihydro-3H-[2,3]benzodiazepines. The latter compounds are obtained from compounds of formula (II) with cyanogen bromide. This nitrile compound is first treated with hydroxylamine and the amidoxime which is obtained is reacted either with a trialkyl orthoformate in the presence of a catalytic amount of hydrochloric acid to give the unsubstituted [1,2,4]oxadiazole derivative or when instead of the orthoformate a carboxylic acid anhydride or chloride is applied then the corresponding (5-alkyl-[1,2,4]oxadiazol-2-yl)-benzodiazepine is formed.

Alternatively, if an above-described intermediate amidoxime type compound is reacted with, for example, 1,1′-carbonyldiimidazole then compounds of formula (I) can be prepared wherein R³ is a (5-oxo-4H-1,2,4-oxadiazol-2-yl) group.

The compounds of formula (I) wherein a 1,2,4-triazolyl group is R³ substituent can be synthesized from a 3-thiocarbamoyl-[2,3]benzodiazepine derivative by reacting with methyl iodide then the obtained S-methyl compound is condensed with hydrazine and the intermediate formed is then treated with a carboxylic acid anhydride or chloride.

Compounds of formula (I), wherein R³ is a (2-oxazolyl) group, can be synthesized by reacting a 3-phenoxycarbonyl-4,5-dihydro-3H-[2,3]benzodiazepine with an amino-ketone-acetal derivative, then the obtained urea derivative, possessing a ketone-acetal side chain on the terminal N-atom, can be brought to ring closure by treatment with a mixture of methanesulfonic acid and phosphorous pentoxide to yield the corresponding 3-(oxazol-2-yl)-4,5-dihydro-3H-[2,3]benzodiazepine.

Other illustrative processes for the synthesis of compounds of formula (I) are those where a hemiketal of formula (VII) is reacted with a heterocyclic reagent substituted with a hydrazine group in the presence of an acid as a catalyst. The condensation reaction can be carried out in the presence of hydrochloric acid as a catalyst by heating with a Dean-Stark apparatus. It can be advantageous in some instances to first transform the hemiketal into an isochromenilium salt of formula (VIIa) with a mineral acid such as perchloric acid and reacting the latter with a hydrazine reagent in isopropanol. The obtained hydrazones of formula (X) are generally formed as a mixture of stereoisomers. They can be further reacted with methanesulfonyl chloride, for example, in dichloromethane in the presence of triethylamine, and the mesylate obtained after isolation can be treated with a concentrated solution of a base in an alcohol or a mixture of alcohol-dichloromethane. The ring closure reaction can be achieved for example, by the Mitsunobu reaction (Mitsunobu, O. Synthesis 1981, 1) as well.

Compounds of formula (I) with a carbamoyl group as the R³ substituent can be synthesized by acylating a compound of formula (II) with an active derivative of a carbamic acid, such as a chloride. Those compounds of formula (I), in which R³ group is R¹¹R¹²NCX, where X stands for an oxygen or sulfur atom, and R¹¹ is hydrogen, can be synthesized conveniently by acylation with an isocyanate or isothiocyanate.

Another synthetic procedure for the preparation of compounds of formula (I) wherein R³ is a carbamoyl group of formula R¹¹R¹²NCO is the following: a compound of formula (II) is reacted first with a phenoxycarbonyl chloride in the presence of an acid binding agent, such as triethylamine, to give a 4,5-dihydro-3-phenoxycarbonyl-3H-[2,3]benzodiazepine derivative. The latter compound is then reacted with a primary or secondary amine to substitute the phenoxy group.

If a compound of formula (I) containing as R³ a group of formula R¹¹R¹²NCS is desired then it can be synthesized from another compound of formula (I), wherein R³ stands for R¹¹R¹²NCO. A thionation reaction can be performed with a Lawesson reagent or phosphorous pentasulfide in an organic solvent.

Compounds of formula (I) wherein R³ is a group of formula R¹³o—CO, wherein R¹³ is an alkyl or phenyl group, can be synthesized from compounds of formula (II) by acylation with the corresponding chlorocarbonic acid ester in the presence of an acid binding agent such as triethylamine.

If desired, the compound of formula (I) obtained by different methods can be transformed into other compounds of formula (I) with further reactions. For example the NH group of an N-containing heterocyclic compound can be alkylated by known methods. The latter transformation for example in the case of a triazolyl compound, can be carried out with methyl iodide in the presence of potassium tert-butoxide.

The reduction of the nitro group in the compounds of formula (I) is generally carried out in polar solvents at room temperature or at elevated temperature in the presence of catalysts such as Raney-nickel, platinum or palladium. Besides gaseous hydrogen, other hydrogen sources e.g., hydrazine hydrate, ammonium formate, potassium formate or cyclohexene can also be applied. The nitro group can be reduced, for example, with tin in the presence of an acid or with tin (II) chloride by heating in an alcohol as well. The amino group can be further derivatized by known methods, for example alkylation, acylation or Sandmeyer reaction.

The new 2,3-benzodiazepine atypical antipsychotic agents of formula (I) of the present invention are useful for the treatment of psychotic disorders, including the treatment of schizophrenia and bipolar disorder. The compounds can also be used for treating schizoaffective disorder, schizophreniform disorder, mood disorders with psychotic symptoms, shared psychotic disorders, and brief psychotic disorder. They may improve functioning in patients with dementia or delirium when psychotic symptoms are present. Additional diseases in which these compounds can be used are aggression, substance-induced psychotic disorders, psychotic disorders due to a general medical condition, and personality disorders (borderline).

Hence, the invention provides a method of treating psychotic disorders comprising administering to a subject in need thereof a therapeutically effective amount of a compound of formula (I) or a stereoisomer or a pharmaceutically acceptable salt thereof. A therapeutically effective amount is a dosage of the compound of formula (I) sufficient to provide a medically desirable result. The therapeutically effective amount of a compound of formula (I) is that amount effective to treat the psychotic disorder or to prevent the onset of diseases, such as aggression or mood disorders.

The factors involved in determining an effective amount are well known to those of ordinary skill in the art and can be addressed with no more than routine experimentation. It is generally preferred that a maximum dose of the compounds of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The dosage of the active ingredient depends on the route of administration, the type and severity of the disease as well as the weight and age of the patient. The daily dose for adult patients generally ranges from about 0.1 mg to about 500 mg, preferably from about 1 mg to about 100 mg, in a single dose or divided in several doses.

The classic, first generation antipsychotics (neuroleptics) like chlorpromazine act by direct blocking the D₂ dopamine receptors. They diminish positive symptoms of schizophrenia (conceptual disorganization, delusions, hallucination) effectively but not the negative ones (anhedonia, flat affect, social withdrawal). By direct blocking of the nigrostriatal dopaminergic pathways they induce extrapyramidal side effects.

The second generation “atypical” antipsychotics like clozapine were introduced into clinical practice in an attempt to enhance therapeutical efficacy (i.e. diminishing both positive and negative symptoms) and to decrease the side effects. Their D₂ antagonist character is weaker and they are antagonists of the serotonin (5HT_{2A}) receptors, too. Atypical antipsychotics have reduced risk for extrapyramidal side effects, however, they are too have some (e.g. agranulocytosis induced by clozapine.) Atypical antipsychotics generally induce remarkable weight gain, increase the risk for diabetes and raise cholesterol level. Possibly due to the serotonergic antagonism, they may induce obsessive-compulsive symptoms, too. Depression and anxiety as well as sleep disturbances are common in psychotic patients, therefore, antipsychotics are mostly not used as monotherapy.

Another aspect of the invention is directed to a composition comprising the compounds of formula (I) (e.g. a pharmaceutical composition). This composition may further include a carrier and/or other additives (e.g. the composition may comprise a compound of formula (I) acting as an active pharmaceutical ingredient and a carrier).

The compounds of formula (I) can be formulated in a pharmaceutically acceptable carrier including diluents, excipients, fillers, binders, solvents, etc. (see Remington's Pharmaceutical Sciences, 18^{th} Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a human or other mammal, generally pharmaceutically acceptable carriers are physiologically inert and non-toxic. Formulations of pharmaceutical compositions may contain more than one type of compound of formula (I), as well as any other pharmacologically active ingredient useful for the treatment of the particular conditions, disease, or symptom being treated.

The compositions of the invention can be administered by standard routes (e.g., oral, inhalation, rectal, nasal, topical, including buccal and sublingual, or parenteral, including subcutaneous, intramuscular, intravenous, intradermal, transdermal, and intratracheal). In addition, polymers may be added according to standard methodologies in the art for sustained release of a given compound.

For oral administration, the compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition including the compound of formula (I) may be effected by liquid solutions, suspensions or elixirs, powders, lozenges, micronized particles and osmotic delivery systems.

Formulations suitable for parenteral administration include aqueous and non-aqueous sterile injection solutions which may contain antioxidants, stabilizers, buffers, bacteriostats and solutes which render the formulation isotonic with the blood of the intended recipient; and aqueous and non-aqueous sterile suspensions which may include suspending agents and thickening agents.

It will be appreciated by those of skill in the art that the number of administrations of the compounds according to the invention will vary from patient to patient based on the particular medical status of that patient at any given time.

### SYNTHETIC EXAMPLES

The compounds according to the invention and the process for their preparation are illustrated in detail by the following Examples.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

The novel starting materials of Examples were synthesized as follows.

### General Procedure for the Synthesis of 7,8-dimethoxy (or dialkoxy)-4-methyl-1-(substituted) phenyl-4,5-dihydro-3H-[2,3]benzodiazepine derivatives

Step 1

(S)-, (R)- or (R,S)-3,4-Dimethoxy-(or dialkoxy)phenyl-isopropanol (10.0 mmol) and an equivalent amount of a substituted benzaldehyde derivative were dissolved in 20 ml of toluene, 0.8 ml of concentrated hydrochloric acid was added and the mixture was stirred for 16 h. The toluene solution was decanted from an oily residue which formed during the reaction and the solvent was evaporated. The residue was triturated with ethanol to give a solid which was then recrystallized from ethanol to give the corresponding 6,7-dialkoxy-3-methyl-1-(substituted phenyl)-isochromane derivative.

Step 2

Method A

To a stirred solution of the isochromane derivative of Step 1 in dichloromethane, containing 5% water, 1.5 equiv. of 2,3-dichloro-5,6-dicyano-1,4-benzoquinone was added over 15 min. Stirring was continued for 3 h, when TLC (eluent: hexane-ethyl acetate) showed full conversion. The suspension was filtered and the filtrate was washed several times with 1N sodium hydroxide solution and water. Drying and evaporation gave the crude hemiketal (a stereoisomeric mixture of 6,7-dialkoxy-1-hydroxy-3-methyl-1-(substituted phenyl)-isochromane derivatives) that was used in Step 3.

Method B

The isochromane of Step 1 was dissolved in a tenfold amount of an 8:7 mixture of dimethylformamide and dimethylsulfoxide. The solution was cooled to 5-10° C. and air, enriched with oxygen up to 40%, was bubbled through the solution. Then a 50% solution of sodium hydroxide in water (2.5 equiv.) was added and stirring was continued for 5 h. The reaction mixture was then poured onto a mixture of ice and water containing hydrochloric acid in an equivalent amount with the previously applied sodium hydroxide. The resulting suspension was aged by stirring for some hours and filtered and the solid washed with water. The thus prepared hemiketal was used without drying in the next step.

Step 3

To a solution of hemiketal (10.0 mmol) of Step 2 and 1.33 equiv. of tert-butyl carbazate in a tenfold amount of toluene, 0.12 ml of concentrated hydrochloric acid was added and the mixture was heated to boiling with constant removal of water. After 3-4 h the mixture was extracted with sodium hydrogen carbonate solution and water. After drying and evaporation, a crude stereoisomeric mixture of hydrazones corresponding to general formula (XI) was formed.

Step 4

To a solution of the hydrazone intermediate of Step 3 (ca. 10.0 mmol) in 45 ml of dichloromethane, 1.5 equivalents of triethylamine were added and it was cooled to 0° C. At this temperature 1.2 equivalents of methanesulfonyl chloride were added dropwise. When TLC (eluent: benzene-ethyl acetate (4:1)) showed complete conversion, the mixture was extracted successively with ice water, 1N hydrochloric acid and brine. Drying and evaporation gave a foam which was dissolved in methanol and 1.2 equivalents of a 50% sodium hydroxide solution in water was added drop-wise at 10° C. Stirring was continued for 2-3 h at r.t., then the solution was concentrated to ⅓ of its volume and water was added to bring the precipitation to completion. After dilution of the reaction mixture with water, sometimes extraction was necessary to isolate the product of the ring closure. The obtained crude 7,8-dialkoxy-3-tert-butoxycarbonyl-4-methyl-4,5-dihydro-3H-[2,3]benzodiazepine derivative was purified by recrystallization or by column chromatography.

Step 5

The tert-butoxycarbonyl-2,3-benzodiazepine derivative of Step 4 was added gradually at room temperature to a six-fold amount of stirred ethyl acetate containing about 13% hydrochloric acid. After 20 minutes generally a suspension formed which was stirred for 3 h. The mixture was then diluted with ethyl acetate and extracted with water, sodium hydrogen carbonate solution and brine. After drying and evaporation the residue was recrystallized to give the title compounds I-XVIII as follows (yields are overall yields).
(R,S)-7,8-Dimethoxy-4-methyl-1-(4-nitrophenyl)-4,5-dihydro-3H-[2,3]benzodiazepine (I), Mp.: 182-183° C., yield: 46%.
(R)-7,8-Dimethoxy-4-methyl-1-(4-nitrophenyl)-4,5-dihydro-3H-[2,3]benzodiazepine (II), Mp.: 171-172° C., yield: 38%, [α]D: +77° (c=0.5, CHCl₃).
(S)-7,8-Dimethoxy-4-methyl-1-(4-nitrophenyl)-4,5-dihydro-3H-[2,3]benzodiazepine (III), Mp.: 168-170° C., yield: 35%, [α]_{D}: −76° (c=0.5, CHCl₃).
(R,S)-7,8-Dimethoxy-4-methyl-1-(2-nitrophenyl)-4,5-dihydro-3H-[2,3]benzodiazepine (IV), Mp.: 111-113° C., yield: 52%.
(R,S)-7,8-Dimethoxy-4-methyl-1-(3-nitrophenyl)-4,5-dihydro-3H-[2,3]benzodiazepine (V), Mp.: 200-202° C., yield: 54%.
(R)-7,8-Dimethoxy-4-methyl-1-(3-nitrophenyl)-4,5-dihydro-3H-[2,3]benzodiazepine (VI), Mp.: 187-188° C., yield: 48%.
(R,S)-7,8-Dimethoxy-4-methyl-1-(3-methyl-4-nitrophenyl)-4,5-dihydro-3H-[2,3]benzodiazepine (VII), Mp.: 154-156° C., yield: 54%.
(R)-7,8-Dimethoxy-4-methyl-1-(3-methyl-4-nitrophenyl)-4,5-dihydro-3H-[2,3]benzodiazepine (VIII), Mp.: 151-152° C., yield: 49%, [α]D: +100° (c=0.5, CHCl₃).
(S)-7,8-Dimethoxy-4-methyl-1-(3-methyl-4-nitrophenyl)-4,5-dihydro-3H-[2,3]benzodiazepine (IX), Mp.: 148-150° C., yield: 46%, [α]_{D}: −99° (c=0.5, CHCl₃).
(R,S)-7,8-Dimethoxy-1-(3-chloro-4-nitrophenyl)-4-methyl-4,5-dihydro-3H-[2,3]benzodiazepine (X), Mp.: 156-158° C., yield: 53%.
(R)-7,8-Dimethoxy-1-(3-chloro-4-nitrophenyl)-4-methyl-4,5-dihydro-3H-[2,3]benzodiazepine (XI), Mp.: 153-154° C., yield: 53%, [α]_{D}: −23° (c=0.5, CHCl₃).
(R)-1-(3,5-Dimethyl-4-nitrophenyl)-7,8-dimethoxy-4-methyl-4,5-dihydro-3H-[2,3]benzodiazepine (XII), Mp.: 186-187° C., yield: 43%, [α]_{D}: +92° (c=0.1, CHCl₃).
(R,S)-7,8-Dimethoxy-1-(3-chlorophenyl)-4-methyl-4,5-dihydro-3H-[2,3]benzodiazepine (XIII), Mp.: 121-122° C., yield: 51%.
(R,S)-7,8-Dimethoxy-1-(4-chlorophenyl)-4-methyl-4,5-dihydro-3H-[2,3]benzodiazepine (XIV), Mp.: 137-140° C., yield: 56%.
(R,S)-7,8-Dimethoxy-1-(4-fluorophenyl)-4-methyl-4,5-dihydro-3H-[2,3]benzodiazepine (XV), Mp.: 136-137° C., yield: 51%.
(R,S)-7,8-Dimethoxy-1-(3,4-dimethoxyphenyl)-4-methyl-4,5-dihydro-3H-[2,3]benzodiazepine (XVI), Mp.: 125-126° C., yield: 55%.
(R,S)-7,8-Dimethoxy-4-methyl-1-phenyl-4,5-dihydro-3H-[2,3]benzodiazepine (XVII), Mp.: 142-145° C., yield: 51%.
(R,S)-7,8-Diethoxy-4-methyl-1-(4-nitrophenyl)-4,5-dihydro-3H-[2,3]benzodiazepine (XVIII), Mp.: 137-138° C., yield: 62%.

### (R,S)-7,8-Dimethoxy-4-methyl-1-(4-nitrophenyl)-3-thiocarbamoyl-4,5-dihydro-3H-[2,3]benzodiazepine (XIX)

A mixture of compound I (2.0 g, 5.86 mmol) and 0.85 (8.75 mmol) potassium thiocyanate in 40 ml of acetic acid was heated at 110° C. for 6 h. After cooling the separated crystals were filtered and washed with water and dried to give 1.94 g (83%) of the title compound. Mp.: 246-247° C.

Compounds XX-XXIV were synthesized similarly to compound XIX. (Occasionally no spontaneous precipitate forming occurred during cooling. In such instances, water was added to the reaction mixture until crystallization began.)

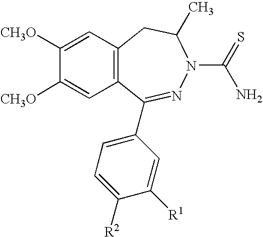

| | Configuration | | | | Yield (%) |
|---|---|---|---|---|---|
| No. | at C-4 | R¹ | R² | Mp (° C.) | [α]_{D} |
| XX | R | H | NO₂ | 242-243 | 77 |
| | | | | | −192° |
| | | | | | (c = 0.3, |
| | | | | | CHCl₃) |
| XXI | R,S | CH₃ | NO₂ | 209-211 | 63 |
| XXII | R | CH₃ | NO₂ | 195-197 | 83 |
| | | | | | −178° |
| | | | | | (c = 0.5, |
| | | | | | CHCl₃) |
| XXIII | R | Cl | NO₂ | 197-198 | 80 |
| | | | | | −156° |
| | | | | | (c = 0.5, |
| | | | | | CHCl₃) |
| XXIV | R,S | Cl | H | 179-180 | 76 |

### (R,S)-7,8-Dimethoxy-4-methyl-1-(4-nitrophenyl)-4,5-dihydro-3H-[2,3]benzodiazepine-3-carbothioyl chloride (XXV)

A solution of 3.41 g (10.0 mmol) of compound I and ml (15.5 mmol) triethylamine in 180 ml of benzene was added drop-wise to a stirred solution of 1.38 g (12.0 mmol) of thiophosgene in 30 ml of benzene. After stirring at r.t. for 16 h the reaction mixture was quenched with water (30 ml). After separation the organic phase was extracted with water (2×30 ml) and brine. After drying and evaporation the residue was triturated with diisopropyl ether to give 3.75 g (89%) of the solid title compound. Mp.: 165-167° C.

Compounds XXVI-XXXV were prepared in a similar fashion:

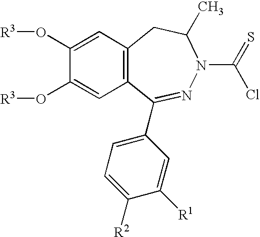

| | Configu- | | | | | |
|---|---|---|---|---|---|---|
| | ration | | | | Mp | Yield (%) |
| No. | at C-4 | R¹ | R² | R³ | (° C.) | [α]_{D} |
| XXVI | R | H | NO₂ | CH₃ | 159-162 | 82 |
| | | | | | | −653° |
| | | | | | | (c = 0.5, |
| | | | | | | CHCl₃) |
| XXVII | S | H | NO₂ | CH₃ | 156-158 | 83 |
| | | | | | | +588° |
| | | | | | | (c = 0.5, |
| | | | | | | CHCl₃) |
| XXVIII | R,S | CH₃ | NO₂ | CH₃ | 174-176 | 85 |
| XXIX | R | CH₃ | NO₂ | CH₃ | 168-170 | 89 |
| | | | | | | −488° |
| | | | | | | (c = 0.5, |
| | | | | | | CHCl₃) |
| XXX | R,S | Cl | NO₂ | CH₃ | 155-157 | 84 |
| XXXI | R | Cl | NO₂ | CH₃ | 151-153 | 87 |
| | | | | | | −526° |
| | | | | | | (c = 0.5, |
| | | | | | | CHCl₃) |
| XXXII | R,S | Cl | H | CH₃ | 77-78 | 79 |
| XXXIII | R,S | H | Cl | CH₃ | 177-178 | 81 |
| XXXIV | R,S | CH₃O | CH₃O | CH₃ | 205-206 | 74 |
| XXXV | R,S | H | NO₂ | CH₃CH₂ | 95-98 | 82 |

### (R,S)-7,8-Dimethoxy-4-methyl-1-(4-nitrophenyl)-4,5-dihydro-3H-[2,3]benzodiazepine-3-carbonitrile (XXXVI)

A mixture of compound I (3.41 g, 10.0 mmol), 1.38 g (10.0 mmol) of dry potassium carbonate and 1.80 g (17.0 mmol) of cyanogen bromide in 50 ml of dimethylformamide was stirred at r.t. for 20 h. The reaction mixture was poured onto water and the precipitate was filtered and washed with water. After drying the title substance weighed 3.45 g (94%). Mp.: 208-211° C.

Similarly, were prepared the following compounds were prepared (starting materials) XXXVII-XLVI:

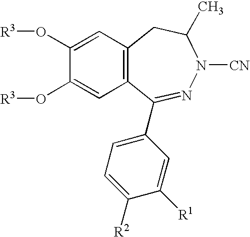

| | Configu- | | | | | |
|---|---|---|---|---|---|---|
| | ration | | | | Mp | Yield (%) |
| No. | at C-4 | R¹ | R² | R³ | (° C.) | [α]_{D} |
| XXXVII | R | H | NO₂ | CH₃ | 187-189 | 92 |
| | | | | | | +46° |
| | | | | | | (c = 0.3, |
| | | | | | | CHCl₃ |
| XXXVIII | R,S | CH₃ | NO₂ | CH₃ | 180-182 | 94 |
| XXXIX | R | CH₃ | NO₂ | CH₃ | 167-169 | 95 |
| | | | | | | +37° |
| | | | | | | (c = 0.2, |
| | | | | | | CHCl₃ |
| XL | S | CH₃ | NO₂ | CH₃ | 175-176 | 84 |
| | | | | | | −41° |
| | | | | | | (c = 0.3, |
| | | | | | | CHCl₃ |
| XLI | R,S | Cl | NO₂ | CH₃ | 160-162 | 89 |
| XLII | R | Cl | NO₂ | CH₃ | 150-155 | 91 |
| | | | | | | +21° |
| | | | | | | (c = 0.5, |
| | | | | | | CHCl₃ |
| XLIII | R,S | H | Cl | CH₃ | 179-180 | 81 |
| XLIV | R,S | H | F | CH₃ | 155-156 | 81 |
| XLV | R,S | CH₃O | CH₃O | CH₃ | 197-198 | 78 |
| XLVI | R,S | H | NO₂ | CH₃CH₂ | 131-132 | 82 |

### Examples 1-11

### General Procedure for the Synthesis of 7,8-dimethoxy-4-methyl-1-(substituted) phenyl-3-(thiazol-2-yl)-4,5-dihydro-3H-[2,3]benzodiazepines

A 7,8-dimethoxy-4-methyl-1-(substituted) phenyl-3-thiocarbamoyl-4,5-dihydro-3H-[2,3]benzodiazepine derivative (selected from corresponding compounds XIX-XXIV) (3.00 mmol) was reacted with a two- to four fold excess of an α-haloaldehyde acetal derivative (bromoacetaldehyde diethyl acetal, 2-bromopropionaldehyde dimethyl acetal) or alternatively with an α-haloketone (chloroacetone, 3-chlorobutanone) in dimethylformamide (12-15 ml) at 80° C. (with the haloaldehyde acetals) or at 40-70° C. (with the haloketones) for 1.5-4 h. Dilution with water resulted in a precipitate which was purified either by recrystallization or column chromatography using silica gel and a (1:1) solvent mixture of hexane and ethyl acetate as eluent or by boiling a suspension of the crude product in ethanol.

The following compounds were prepared by this procedure:

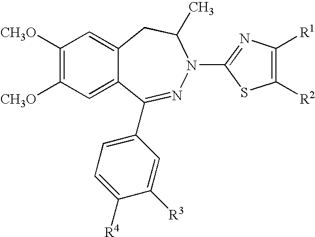

| | Configu- | | | | | | |
|---|---|---|---|---|---|---|---|
| No. of | ration | | | | | Mp | Yield (%) |
| Example | at C-4 | R¹ | R² | R³ | R⁴ | (° C.) | [α]_{D} |
| 1 | R,S | H | H | H | NO₂ | 195-197 | 75 |
| 2 | R | H | H | H | NO₂ | 151-152 | 74 |
| | | | | | | | +663° |
| | | | | | | | (c = 0.5, |
| | | | | | | | CHCl₃ |
| 3 | R,S | H | H | CH₃ | NO₂ | 145-147 | 79 |
| 4 | R | H | H | CH₃ | NO₂ | 172-174 | 73 |
| | | | | | | | +578° |
| | | | | | | | (c = 0.5, |
| | | | | | | | CHCl₃ |
| 5 | R | H | H | Cl | NO₂ | 168-169 | 76 |
| | | | | | | | +766° |
| | | | | | | | (c = 0.5, |
| | | | | | | | CHCl₃ |
| 6 | R,S | CH₃ | H | H | NO₂ | 194-195 | 87 |
| 7 | R,S | H | CH₃ | H | NO₂ | 122-123 | 90 |
| 8 | R,S | CH₃ | CH₃ | H | NO₂ | 192-193 | 96 |
| 9 | R | CH₃ | CH₃ | H | NO₂ | 190-192 | 84 |
| | | | | | | | +414° |
| | | | | | | | (c = 0.2, |
| | | | | | | | CHCl₃ |
| 10 | R,S | H | H | Cl | H | 125-126 | 75 |
| 11 | R,S | CH₃ | CH₃ | Cl | H | 174-176 | 78 |
| | | | | | | (HCl | |
| | | | | | | salt) | |

### Example 12

### (R,S)-3-(4,5-Dihydro-thiazol-2-yl)-7,8-dimethoxy-4-methyl-1-(4-nitrophenyl)-4,5-dihydro-2H-[2,3]benzodiazepine

Starting compound XIX (1.00 g, 2.45 mmol) was heated with 1.20 g (5.86 mmol) of 2-bromoethylamine hydrobromide in 20 ml of dimethylformamide at 70° C. for 12 h. After dilution with water the precipitate was filtered and recrystallized from ethanol to give 0.83 g (79%) of title compound. Mp.: 220-222° C.

### Example 13

### (R)-3-(4,5-Dihydro-thiazol-2-yl)-7,8-dimethoxy-4-methyl-1-(4-nitrophenyl)-4,5-dihydro-2H-[2,3]benzodiazepine

Starting compound XX was reacted as in Example 13 to form the title compound.

Yield: 79%, mp.: 206-212° C. [α]_{D}: +714° (c=0.2, CHCl₃)

### Example 14

### (R,S)-3-(4,5-Dihydro-4-oxo-thiazol-2-yl)-7,8-dimethoxy-4-methyl-1-(4-nitrophenyl)-4,5-dihydro-2H-[2,3]benzodiazepine

Starting compound XIX (0.50 g, 1.25 mmol) was reacted with 0.38 g (2.48 mmol) of methyl-2-bromoacetate in 14 ml of dimethylformamide at 50° C. for 2 h. The reaction mixture was diluted with water and the precipitate formed was filtered and dried. Purification of this product was done by stirring and heating an ethanolic suspension for 15 min at boiling temperature. Filtration gave the title compound (0.52 g, 94%), mp.: 238-239° C.

### Example 15

### (R,S)-3-(4,5-Dihydro-5-methyl-4-oxo-thiazol-2-yl)-7,8-dimethoxy-4-methyl-1-(4-nitrophenyl)-4,5-dihydro-2H-[2,3]benzodiazepine

The title compound was prepared as in example 14, but with ethyl-2-bromopropionate as a reagent.

Yield: 92%, mp.: 213-214° C.

### Examples 16-28

### General Procedure for Synthesis of 7,8-dialkoxy-4-methyl-1-(substituted) phenyl-3-(1,3,4-thiadiazol-2-yl)-4,5-dihydro-3H-[2,3]benzodiazepines

A 4,5-dihydro-3H-[2,3]benzodiazepine-3-carbothioyl chloride derivative (one of starting compounds XXV-XXXV) (8.1 mmol) was added gradually to a vigorously stirred mixture of 1.21 g (24.2 mmol) of 98% hydrazine hydrate in tetrahydrofuran (72 ml) at 5-10° C. After 1 h stirring at r.t., the solvent was evaporated and the residue triturated with water. The precipitate was filtered and dried. The thus prepared intermediate 4,5-dihydro-3H-[2,3]benzodiazepine-3-carbothiohydrazide derivative was heated in 30 ml of triethyl orthoformate at 100° C. for 2 h. After cooling a precipitate formed which was filtered and washed with ethanol to give the title products with a non-substituted 1,3,4-thiadiazole ring as substituent.

Alternatively, in the examples in which a 5′-methyl or ethyl substituted 3-(1,3,4-thiadiazol-2-yl)benzodiazepine was formed, the intermediate 3-carbothiohydrazide derivative was first reacted at r.t. with 1.2 equiv. of acetylchloride in dichloromethane in the presence of triethylamine or was reacted with 1.5 equiv. of propionic anhydride at r.t. for 3 h and then p-toluenesulfonic acid hydrate (1.5 equiv) was added and the mixture was stirred for 16 h. After dilution with dichloromethane the solution was extracted successively with water, sodium hydrogencarbonate and water. The organic phase was dried and the solvent evaporated to give the title product, which was purified by recrystallization from ethanol.

The following compounds were prepared according to this procedure:

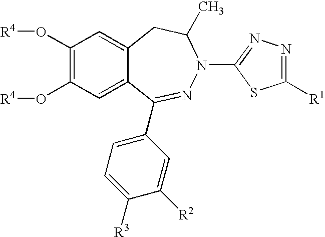

| No. | | | | | | | |
|---|---|---|---|---|---|---|---|
| of | | | | | | | |
| Ex- | Config- | | | | | | |
| am- | uration | | | | | Mp | Yield (%) |
| ple | at C-4 | R¹ | R² | R³ | R⁴ | (° C.) | [α]_{D} |
| 16 | R,S | H | H | NO₂ | CH₃ | 210-212 | 74 |
| 17 | R | H | H | NO₂ | CH₃ | 224-225 | 66 |
| | | | | | | | +610° |
| | | | | | | | (c = 0.5, |
| | | | | | | | CHCl₃) |
| 18 | S | H | H | NO₂ | CH₃ | 229-231 | 68 |
| | | | | | | | −549° |
| | | | | | | | (c = 0.5, |
| | | | | | | | CHCl₃) |
| 19 | R,S | H | CH₃ | NO₂ | CH₃ | 195-196 | 80 |
| 20 | R | H | CH₃ | NO₂ | CH₃ | 192-195 | 74 |
| | | | | | | | +429° |
| | | | | | | | (c = 0.1, |
| | | | | | | | CHCl₃) |
| 21 | R,S | H | Cl | NO₂ | CH₃ | 214-215 | 71 |
| 22 | R | H | Cl | NO₂ | CH₃ | 203-206 | 67 |
| | | | | | | | +613° |
| | | | | | | | (c = 0.3, |
| | | | | | | | CHCl₃) |
| 23 | R,S | H | Cl | H | CH₃ | 86-88 | 65 |
| 24 | R,S | H | H | Cl | CH₃ | 203-204 | 68 |
| 25 | R,S | H | CH₃O | CH₃O | CH₃ | 173-174 | 59 |
| 26 | R,S | H | H | NO₂ | CH₃CH₂ | 172-174 | 68 |
| 27 | R,S | CH₃ | H | NO₂ | CH₃ | 228-229 | 84 |
| 28 | R | CH₃ | H | NO₂ | CH₃ | 203-204 | 85 |
| | | CH₂ | | | | | |

### Examples 29, 30

### General Procedure for the Synthesis of 7,8-dimethoxy-4-methyl-1-(4-nitrophenyl)-3-(1,3,4-oxadiazol-2-yl)-4,5-dihydro-3H-[2,3]benzodiazepines

Step 1

To a solution of starting compound XXV (0.78 g, 1.86 mmol), 0.26 ml (1.87 mmol) of triethylamine and a catalytic amount of 4-dimethylaminopyridine in 10 ml of dimethylformamide formic hydrazide (or acethydrazide) (18.6 mmol) dissolved in dimethylformamide (10 ml) was added drop-wise. The reaction mixture was stirred at r.t. for 70 h, then diluted with water and the precipitate was filtered. This intermediate was purified by column chromatography (silica gel, eluent: hexane-ethyl acetate (1:2)).

Step 2

The intermediate of Step 1 was dissolved in ethanol (10 ml) and after the addition of mercury (II) acetate (0.38 g, 1.19 mmol) the mixture was stirred and heated to a boil for 2 h. After evaporation of the solvent the residue was taken up with dichloromethane and filtered through a pad of neutral aluminium oxide. The solution was evaporated to dryness and the residue was purified by column chromatography (silica gel, eluent: hexane-ethyl acetate (2:3)).

The following compounds were prepared by this procedure:

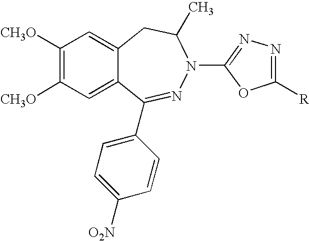

| No. of | Configuration | | | |
|---|---|---|---|---|
| Example | at C-4 | R | Mp (° C.) | Yield (%) |
| 29 | R,S | H | 225-229 | 33 |
| 30 | R,S | CH₃ | 177-178 | 41 |

### Example 31

### (R,S)-7,8-Dimethoxy-4-methyl-1-(4-nitrophenyl)-3-(5-oxo-5,6-dihydro-4H-[1,3,4]thiadiazin-2-yl)-4,5-dihydro-3H-[2,3]benzodiazepine

Starting compound XXV was transformed into the corresponding 3-carbothiohydrazide intermediate according to the general procedure described for Examples 16-28. A mixture of this intermediate (0.83 g, 2.0 mmol), 0.34 g (2.46 mmol) of dry potassium carbonate and 0.20 ml (2.45 mmol) of chloroacetyl chloride in 10 ml of dimethylformamide was then stirred for 24 h. The reaction mixture was quenched with a five-fold amount of water and the precipitate was filtered, washed with water and dried. The crude product was purified by column chromatography using ethyl acetate-hexane (1:1) as an eluent. The title substance weighed 0.57 g (62%), mp.: 229-231° C.

### Examples 32-37

### General Procedure for Synthesis of 7,8-dimethoxy-4-methyl-1-(substituted) phenyl-3-(1,4,2-oxathiazol-3-yl)-4,5-dihydro-3H-[2,3]benzodiazepines

The corresponding starting compound carbothioyl chloride from the group of XXV-XXXV (3.86 mmol) was added gradually over 15 min to a stirred and cooled (0-5° C.) mixture of 0.82 g (11.8 mmol) hydroxylamine hydrochloride and 0.49 g (12.25 mmol) sodium hydroxide in 23 ml of ethanol. Stirring was continued at r.t. for 20 h and after dilution with water the precipitate was filtered and dried. This intermediate thiohydroxamic acid was dissolved in acetone and potassium carbonate (0.69 g, 4.99 mmol) was added. To this suspension a solution of diiodomethane (1.1 g, 4.11 mmol) in 5 ml of acetone was added drop-wise and stirring was continued for 24 h. The reaction mixture was diluted with water and extracted with ethyl acetate. Evaporation gave the crude product which was purified by column chromatography (silica gel, eluent: hexane-ethyl:acetate (1:1)).

The following compounds were prepared according to the above procedure:

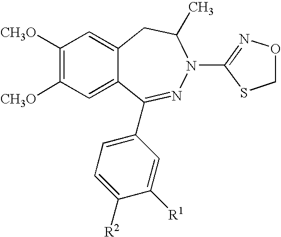

| No. of | Configuration | | | | Yield (%) |
|---|---|---|---|---|---|
| Example | at C-4 | R¹ | R² | Mp (° C) | [α]_{D} |
| 32 | R,S | H | NO₂ | 195-198 | 38 |
| 33 | R | H | NO₂ | 206-208 | 31 |
| | | | | | +604° |
| | | | | | (c = 0.5, |
| | | | | | CHCl₃) |
| 34 | S | H | NO₂ | 209-211 | 36 |
| | | | | | −551° |
| | | | | | (c = 0.5, |
| | | | | | CHCl₃) |
| 35 | R | CH₃ | NO₂ | 180-182 | 35 |
| | | | | | +429° |
| | | | | | (c = 0.1, |
| | | | | | CHCl₃) |
| 36 | R,S | H | F | 186-191 | 43 |
| 37 | R,S | H | H | 170-173 | 38 |

### Example 38

### (R,S)-Dimethoxy-4-methyl-3-(2-methyl-3-oxo-2,3-dihydro-1,2,4-thiadiazol-5-yl)-1-(4-nitrophenyl)-4,5-dihydro-[2,3]benzodiazepine

Step 1

To a solution of 0.37 g (3.80 mmol) of potassium thiocyanate in 8 ml of acetone, phenyl chloroformate (0.48 ml, 3.80 mmol) was added drop-wise at r.t. Stirring was continued at r.t. for 30 min and then it was heated to 40° C. for 15 min. This solution was cooled with ice-water and a solution of the starting compound I (1.09 g, 3.19 mmol) in acetone (15 ml) was added drop-wise. Stirring was continued at r.t. for 20 h and the mixture was poured onto water. The 3-(phenoxycarbonyl-thiocarbamoyl)-benzodiazepine intermediate separated as a precipitate, which was filtered, washed and dried to give a solid with mp.: 107-110° C.

Step 2

This solid intermediate was dissolved in 10 ml of dimethylformamide and a methylamine solution in water (40%, 0.35 ml, 4.04 mmol) was added drop-wise. After 3 h of stirring, the mixture was diluted with water and the precipitate formed was isolated by suction, washed and dried. The thus formed intermediate: (R,S)-1-methyl-3-[7,8-dimethoxy-4-methyl-1-(4-nitrophenyl)-4,5-dihydro-3H-[2,3]benzodiazepine-3-carbothioyl]-urea (mp.: 197-199° C.) was used in the next ring-closure step.

Step 3

The compound prepared in Step 2 (1.01 g, 2.21 mmol) was dissolved in chloroform (20 ml) and a solution of bromine (0.42 g, 2.63 mmol) in chloroform (6 ml) was added drop-wise. After 1 h, the solution was diluted with 30 ml of chloroform and extracted successively with sodium bicarbonate solution and water. After drying and evaporation the remaining crude title product was purified by column chromatography on silica gel applying a 95:5 mixture of ethyl acetate as an eluent. Evaporation of the main fraction gave the title product: 0.75 g (56% overall). Mp.: 262-263° C.

### Example 39

### (R,S)-7,8-Dimethoxy-3-(4,5-dihydro-oxazol-2-yl)-4-methyl-1-(4-nitrophenyl)-4,5-dihydro-3H-[2,3]benzodiazepine

Starting substance I (1.50 g, 4.39 mmol) was reacted with 2-chloroethyl isocyanate (0.93 g, 8.82 mmol) in dichloromethane (30 ml) for 24 h at r.t. After evaporation the residue washed with hexane and dissolved in 30 ml of dimethylformamide. Dry potassium carbonate (0.69 g, 4.99 mmol) and potassium iodide (0.40 g, 2.41 mmol) were added and the mixture was heated at 100-110° C. for 6 h. Dilution with water gave a solid which was purified by column chromatography on silica gel using a 95:5 mixture of ethyl acetate and methanol as eluent to give 0.62 g (34%) of the title product as a solid foam.

### Examples 40-53

### General Procedure for Synthesis of 7,8-dialkoxy-4-methyl-1-(substituted) phenyl-3-(1,2,4-oxadiazol-3-yl)-4,5 dihydro-3H-[2,3]benzodiazepines

To a solution of a benzodiazepine-3-carbonitrile derivative from starting compounds XXXVI-XLVI (11.05 mmol) in tetrahydrofuran 1.60 g (19.5 mmol) of sodium acetate and 0.95 g (13.67 mmol) of hydroxylamine hydrochloride was added and the mixture was stirred at r.t. for 5 h. The solvent was evaporated and the residue treated with water gave the corresponding amidoxime intermediate which was isolated by filtration. After drying this intermediate was taken up in triethyl orthoformate (40 ml), catalytic amount of concentrated hydrochloric acid was added and the mixture was stirred and heated for 1 h at 110° C. (Alternatively, acetic anhydride (10-15 ml) was used instead of triethyl orthoformate for the synthesis of (5-methyl-1,2,4-oxadiazol-3-yl) substituted benzodiazepines.) After cooling, title products usually crystallized out of the reaction mixture or the reaction mixture was concentrated and the residue was treated with water, then the product was extracted with ethyl acetate. Crude products were purified by heating their ethanolic suspensions to boiling and after cooling the crystals were filtered off.

The following compounds were prepared by the above procedure:

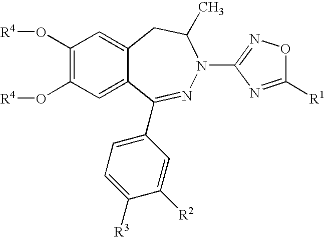

| No. | | | | | | | |
|---|---|---|---|---|---|---|---|
| of | | | | | | | |
| Ex- | Config- | | | | | | |
| am- | uration | | | | | Mp | Yield (%) |
| ple | at C-4 | R¹ | R² | R³ | R⁴ | (° C.) | [α]_{D} |
| 40 | R,S | H | H | NO₂ | CH₃ | 213-214 | 72 |
| 41 | R | H | H | NO₂ | CH₃ | 190-192 | 65 |
| | | | | | | | −409° |
| | | | | | | | (c = 0.5, |
| | | | | | | | CHCl₃) |
| 42 | R,S | H | CH₃ | NO₂ | CH₃ | 229-230 | 88 |
| 43 | R | H | CH₃ | NO₂ | CH₃ | 223-225 | 79 |
| | | | | | | | −483° |
| | | | | | | | (c = 0.3, |
| | | | | | | | CHCl₃) |
| 44 | S | H | CH₃ | NO₂ | CH₃ | 216-218 | 69 |
| | | | | | | | +452° |
| | | | | | | | (c = 0.2, |
| | | | | | | | CHCl₃) |
| 45 | R,S | H | Cl | NO₂ | CH₃ | 227-230 | 71 |
| 46 | R | H | Cl | NO₂ | CH₃ | 206-208 | 70 |
| | | | | | | | −421° |
| | | | | | | | (c = 0.25, |
| | | | | | | | CHCl₃) |
| 47 | R | CH₃ | CH₃ | NO₂ | CH₃ | 206-208 | 65 |
| | | | | | | | −456° |
| | | | | | | | (c = 0.4, |
| | | | | | | | CHCl₃) |
| 48 | R,S | CH₃ | H | NO₂ | CH₃ | 197-199 | 63 |
| 49 | R,S | H | H | Cl | CH₃ | 210-212 | 70 |
| 50 | R,S | H | H | F | CH₃ | 217-220 | 79 |
| 51 | R,S | H | H | H | CH₃ | 215-216 | 78 |
| 52 | R,S | H | H | NO₂ | CH₃CH₂ | 158-162 | 48 |
| 53 | R,S | H | CH₃O | CH₃O | CH₃ | 188-189 | 69 |

### Example 54

### (R)-7,8-Dimethoxy-4-methyl-1-(3-methyl-4-nitrophenyl)-3-(5-oxo-4H-1,2,4-oxadiazol-3-yl)-4,5-dihydro-3H-[2,3]benzodiazepine

Starting compound XXXIX (1.60 g, 4.21 mmol) was transformed into the amidoxime intermediate according to the general procedure described for Examples 40-53. This intermediate (1.38 g, 3.34 mmol) was dissolved in 60 ml of dichloromethane and reacted with 0.59 g (3.64 mmol) of 1,1′-carbonyldiimidazole at 40° C. for 10 h. The reaction mixture was diluted with dichloromethane (60 ml), extracted successively with 0.25N hydrochloric acid and water. After drying and evaporation the crude title product was recrystallized from ethanol to give 1.26 g (68%). Mp.: 221-223° C. [α]_{D}: +242° (c=1.5, CHCl₃).

### Examples 55-60

### General Procedure for Synthesis of 7,8-dimethoxy-4-methyl-1-(4-nitrophenyl)-3-[1H(2H)-1,2,4-triazol-3-yl]-4,5-dihydro-3H-[2,3]benzodiazepines

Step 1

Starting compound XIX was reacted in dimethylformamide at r.t. with an equivalent amount of methyl iodide in the presence of potassium carbonate to give (R,S)-7,8-dimethoxy-4-methyl-1-(4-nitrophenyl)-4,5-dihydro-3H-[2,3]benzodiazepine-3-S-methyl-thiocarboximidate. Mp.: 153-155° C.

Step 2

The compound prepared in Step 1 (0.82 g, 1.98 mmol) was heated in 2-methoxyethanol (30 ml) with formic hydrazide or acethydrazide (19.98 mmol) and a catalytic amount of p-toluenesulfonic acid at 100-110° C. for 5 h. After evaporation the residue was triturated with water to give the crude product which was purified by column chromatography on silica gel with hexane-ethyl acetate (1:2) as an eluent.

Step 3 (Optional)

N-methylated derivatives of the title compounds were prepared from compounds obtained in Step 2 by reacting the latter with methyl iodide in tetrahydrofuran in the presence of equivalent amount of tert-butoxide at r.t. for 16 h. Then the reaction mixture was diluted with water and the products were extracted with ethyl acetate. Two products formed in each of the reactions, corresponding to the tautomeric possibilities, which were separated by column chromatography on silica gel using ethyl acetate as an eluent.

The following compounds were prepared (yields are overall)

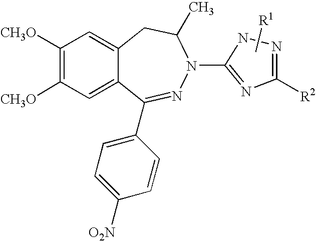

| No. of | Configuration | | | | |
|---|---|---|---|---|---|
| Example | at C-4 | R¹ | R² | Mp (° C.) | Yield (%) |
| 55 | R,S | H | H | 244-246 | 39 |
| 56 | R,S | H | CH₃ | 237-239 | 56 |
| 57 | R,S | 1-CH₃ | H | 132-136 | 23 |
| 58 | R,S | 2-CH₃ | H | 98-100 | 24 |
| 59 | R,S | 1-CH₃ | CH₃ | 250-252 | 19 |
| 60 | R,S | 2-CH₃ | CH₃ | 79-81 | 17 |

### Example 61

### (R,S)-7,8-Dimethoxy-4-methyl-1-(4-nitrophenyl)-3-(5-methyloxazol-2-yl)-4,5-dihydro-3H-[2,3]benzodiazepine

A solution of the compound of Example 63 (3.2 g, 7.35 mmol) and 8.10 g (69.2 mmol) C-(2-methyl-[1,3]dioxolan-2-yl)-methylamine in 200 ml of 1,2-dichloroethane was stored at r.t. for 36 h. The solution was then washed with water (3×100 ml), dried and evaporated to dryness. The thus prepared solid was recrystallized from ethanol to give 2.95 g (83%, mp.: 139.140° C.) of the intermediate (R,S)-7,8-dimethoxy-3-[N′-(2,2-ethylenedioxy-1-propyl)-carbamoyl]-4-methyl-1-(4-nitrophenyl)-4,5-dihydro-[2,3]benzodiazepine.

Of the above intermediate 2.50 g (5.16 mmol) were added gradually to 40 ml of methanesulfonic acid at r.t., followed by the addition of 4.00 g (14.1 mmol) of phosphorous pentoxide. This reaction mixture was kept at r.t. for 9 days and then it was quenched carefully with 400 ml of ice-water and neutralized with potassium carbonate. A solid was separated which was dissolved in dichloromethane. This solution was washed with water, dried and evaporated to dryness. The thus prepared crude title product was recrystallized from methanol to give 0.71 g (36%) of the product with mp.: 161-166° C.

### Example 62

### (R)-7,8-Dimethoxy-4-methyl-1-(4-nitrophenyl)-3-(2-pyridyl)-4,5-dihydro-3H-[2,3]benzodiazepine

Step 1

(3S)-6,7-Dimethoxy-1-hydroxy-3-methyl-1-(4-nitrophenyl)-isochromane (2.40 g, 9.65 mmol; product of Step 2 of general procedure for Starting compound II) and 2-hydrazinopyridine (0.91 g, 8.34 mmol) were dissolved in toluene (30 ml) and after the addition of 0.20 ml of concentrated hydrochloric acid the mixture was heated under reflux with continuous removal of water. After 3 h the mixture was extracted with sodium carbonate solution and water and the solvent was evaporated to give a solid, which was recrystallized from ethanol to give the hydrazone intermediate: 1.54 g (3.54 mmol), mp.: 233-234° C.

Step 2

To a solution of the above hydrazone intermediate in 60 ml of dichloromethane 0.96 ml (6.19 mmol) of triethylamine was added. The solution was cooled to 0° C. Methanesulfonyl chloride (0.45 ml, 5.70 mmol) was added drop-wise and the mixture was stirred at 0-5° C. for 2 h. The reaction mixture was then extracted successively with ice-water, 1N hydrochloric acid and brine and it was concentrated to a volume of about 7 ml. To this mixture 6 ml of methanol were added and a 50% sodium hydroxide solution in water (prepared from 0.20 g (5.0 mmol) of sodium hydroxide and 0.20 ml of water) was added dropwise at ca 10° C. Stirring was continued for 3 h at r.t., then the solution was evaporated to ⅓ of its volume and 30 ml of water was added gradually over 30 min. Crystals separated which were filtered and washed with water. The crude product was recrystallized after drying from 7.2 ml of 2-methoxyethanol to give 1.14 g (28%) of the title product with mp.: 210-214° C.

### Example 63

### (R,S)-7,8-Dimethoxy-4-methyl-1-(4-nitrophenyl)-3-phenoxycarbonyl-4,5-dihydro-3H-[2,3]benzodiazepine

To a solution of starting compound I (3.41 g, 10.0 mmol) in 100 ml of dichloromethane 2.75 ml (20.0 mmol) of triethylamine and 3.13 g (20.0 mmol) of phenyl chloroformate was added and the reaction mixture was kept at r.t. for 24 h. After evaporation of the solvent, the residue was triturated with 60 ml of a 10% sodium hydrogen carbonate solution in water and the precipitate was filtered. After drying, the substance was suspended and heated to boiling in diisopropyl ether for 30 min and filtered while hot. The title compound weighed 4.12 g (89%). Mp.: 201-203° C.

### Example 64

### (R,S)-7,8-Dimethoxy-4-methyl-1-(4-nitrophenyl)-4,5-dihydro-3H-[2,3]benzodiazepine-3-carboxylic acid imidazolide

Starting compound I (3.41 g, 10.0 mmol) was reacted in 75 ml of tetrahydrofuran with 1.95 g (12.0 mmol) of 1,1′-carbonyldiimidazole for 30 min at boiling temperature. The reaction mixture was evaporated to dryness and the residue was recrystallized from ethanol to give 3.71 g (85%) of the title compound. Mp.: 164-165° C.

### Example 65

### (R)-7,8-Dimethoxy-4-methyl-1-(4-nitrophenyl)-4,5-dihydro-3H-[2,3]benzodiazepine-3-carboxylic acid imidazolide

The title compound was prepared as described in example 64, using the appropriate starting compound. Mp.: 161-162° C., yield: 81%; [α]_{D}: −380° (c=0.5, CHCl₃).

### Example 66

### (R)-7,8-Dimethoxy-4-methyl-1-(3-methyl-4-nitrophenyl)-4,5-dihydro-3H-[2,3]benzodiazepine-3-carboxylic acid imidazolide

The title compound was prepared as described in example 64, using the appropriate starting compound. Mp.: 167-169° C., yield: 85%; [α]_{D}: −379° (c=0.3, CHCl₃).

### Examples 67-101

### General Procedures for Synthesis of 3-carbamoyl-1-(substituted) phenyl-4,5-dihydro-3H-[2,3]benzodiazepines

The compounds of examples 67-101 were prepared using one of the 5 methods below.

Method A

A 3-unsubstituted dihydro-benzodiazepine from the group of starting materials I-XVIII (1.99 mmol) was reacted in dichloromethane (10 ml) with an excess of an alkyl isocyanate (12.09 mmol) at r.t. for 48 h. After evaporation the residue was triturated with water and the precipitate was filtered and washed with water. The crude substance was purified by recrystallization from ethanol or column chromatography (silica gel, eluent: hexane-ethyl acetate (1:1)).

Method B

A mixture of 1.99 mmol of a starting compound from the group I-XVIII, 0.28 g (0.24 mmol) of potassium carbonate and 1.29 g (12.0 mmol) of dimethylcarbamoyl chloride was stirred at r.t. for 30 h. The residue after evaporation was triturated with water and the solid was filtered. The crude product was purified by column chromatography on silica gel using hexane-ethyl acetate (1:1) as an eluent.

Method C

The compound of example 63 (0.90 g, 1.95 mmol) was reacted in dimethylformamide (23 ml) with about a tenfold excess of a secondary or primary amine at 90-120° C. for 4-7 h. The reaction mixture was poured onto water and the solid product formed was isolated by filtration. If necessary the product was recrystallized.

Method D

A compound of Examples 64-66 (1.0 mmol) was reacted in tetrahydrofuran or ethanol (15 ml) in the presence of 1,8-diazabicyclo[5.4.0]undec-7-ene (0.05 mmol) with the corresponding alkylamine (8.5-20 mmol) by heating at reflux temperature for 3-6 h. The reaction mixture was then concentrated to dryness and the residue was taken up with water. The filtered and dried product was recrystallized from ethanol or purified by column chromatography on silica gel using hexane-ethylacetate mixtures as eluent.

Method E

To a stirred mixture of O-methylhydroxylamine hydrochloride g, 13.17 mmol) in dimethylsulfoxide (55 ml) 1.8-diazabicyclo[5,4,0]undec-7-ene (2.75 g, 18.06 mmol) was added and stirring was continued for 0-5 h. To this solution 2.53 mmol of a compound of examples 64 or 65 was added and the reaction was stored at r.t. for 24 h. After dilution with water the mixture was extracted with ethyl acetate and evaporation of the extract gave the product which was purified by column chromatography on silica gel using hexane-ethyl acetate (1:1) as eluent.

The following compounds were prepared by the above procedures:

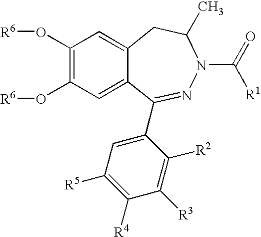

| | Configura- | Method | | | | | | | | |
|---|---|---|---|---|---|---|---|---|---|---|
| No. of | tion at | of | | | | | | | | Yield (%) |
| Example | C-4 | synthesis | R¹ | R² | R³ | R⁴ | R⁵ | R⁶ | Mp (° C.) | [α]_{D} |
| 67 | R,S | A,D | CH₃NH | H | H | NO₂ | H | CH₃ | 196-197 | 74 |
| 68 | R,S | A | CH₃NH | NO₂ | H | H | H | CH₃ | 174-176 | 76 |
| 69 | R,S | A | CH₃NH | H | NO₂ | H | H | CH₃ | 83-85 | 74 |
| 70 | R | A,D | CH₃NH | H | H | NO₂ | H | CH₃ | 134-135 | 76 |
| | | | | | | | | | | +320° |
| | | | | | | | | | | (c = 0.5, |
| | | | | | | | | | | CHCl₃) |
| 71 | S | A | CH₃NH | H | H | NO₂ | H | CH₃ | 117-123 | 83 |
| | | | | | | | | | | −325° |
| | | | | | | | | | | (c = 0.2, |
| | | | | | | | | | | CHCl₃) |
| 72 | R,S | A,D | CH₃NH | H | CH₃ | NO₂ | H | CH₃ | 194-195 | 88 |
| 73 | R | A | CH₃NH | H | CH₃ | NO₂ | H | CH₃ | 177-179 | 81 |
| | | | | | | | | | | +339° |
| | | | | | | | | | | (c = 0.5, |
| | | | | | | | | | | CHCl₃) |
| 74 | S | A | CH₃NH | H | CH₃ | NO₂ | H | CH₃ | 177-178 | 75 |
| | | | | | | | | | | −344° |
| | | | | | | | | | | (c = 0.5, |
| | | | | | | | | | | CHCl₃) |
| 75 | R,S | A | CH₃NH | H | Cl | NO₂ | H | CH₃ | 194-195 | 85 |
| 76 | R | A | CH₃NH | H | Cl | NO₂ | H | CH₃ | 195-197 | 79 |
| | | | | | | | | | | +360° |
| | | | | | | | | | | (c = 0.5, |
| | | | | | | | | | | CHCl₃) |
| 77 | R | A | CH₃NH | H | CH₃ | NO₂ | CH₃ | CH₃ | 158-160 | 87 |
| | | | | | | | | | | +249° |
| | | | | | | | | | | (c = 0.3, |
| | | | | | | | | | | CHCl₃) |
| 78 | R,S | A | CH₃CH₂NH | H | H | NO₂ | H | CH₃ | 206-208 | 88 |
| 79 | R | A | CH₃CH₂NH | H | H | NO₂ | H | CH₃ | 186-189 | 69 |
| | | | | | | | | | | +400° |
| | | | | | | | | | | (c = 0.5, |
| | | | | | | | | | | CHCl₃) |
| 80 | R | A | CH₃CH₂NH | H | CH₃ | NO₂ | H | CH₃ | 149-150 | 74 |
| | | | | | | | | | | +369° |
| | | | | | | | | | | (c = 0.3, |
| | | | | | | | | | | CHCl₃) |
| 81 | R,S | A | (CH₃)₂CHNH | H | H | NO₂ | H | CH₃ | 177-178 | 90 |
| 82 | R | A | (CH₃)₂CHNH | H | H | NO₂ | H | CH₃ | 117-119 | 68 |
| | | | | | | | | | | +395° |
| | | | | | | | | | | (c = 0.2, |
| | | | | | | | | | | CHCl₃) |
| 83 | R | A | (CH₃)₂CHNH | H | CH₃ | NO₂ | H | CH₃ | 127-128 | 71 |
| | | | | | | | | | | +414° |
| | | | | | | | | | | (c = 0.3, |
| | | | | | | | | | | CHCl₃) |
| 84 | R | D | CH₃(CH₂)₂NH | H | H | NO₂ | H | CH₃ | 135-136 | 75 |
| | | | | | | | | | | +363° |
| | | | | | | | | | | (c = 0.2, |
| | | | | | | | | | | CHCl₃) |
| 85 | R | D | CH₃(CH₂)₂NH | H | CH₃ | NO₂ | H | CH₃ | 143-144 | 81 |
| | | | | | | | | | | +375° |
| | | | | | | | | | | (c = 0.2, |
| | | | | | | | | | | CHCl₃) |
| 86 | R,S | D | CH₃(CH₂)₃NH | H | H | NO₂ | H | CH₃ | 148-152 | 91 |
| 87 | R | A | C₆H₅NH | H | CH₃ | NO₂ | H | CH₃ | 147-148 | 85 |
| | | | | | | | | | | +362° |
| | | | | | | | | | | (c = 0.3, |
| | | | | | | | | | | CHCl₃) |
| 88 | R,S | A | CH₃NH | H | Cl | H | H | CH₃ | 90-91 | 76 |
| 89 | R,S | A | CH₃NH | H | H | Cl | H | CH₃ | 164-166 | 78 |
| 90 | R,S | A | CH₃NH | H | H | F | H | CH₃ | 80-83 | 64 |
| 91 | R | B | (CH₃)₂N | H | H | NO₂ | H | CH₃ | 92-96 | 74 |
| | | | | | | | | | | +159° |
| | | | | | | | | | | (c = 0.3, |
| | | | | | | | | | | CHCl₃) |
| 92 | R,S | A | CH₃NH | H | H | H | H | CH₃ | 86-88 | 69 |
| 93 | R,S | A | CH₃NH | H | H | NO₂ | H | CH₃CH₂ | 188-189 | 75 |
| 94 | R,S | A | CH₃NH | H | CH₃O | CH₃O | H | CH₃ | 185-186 | 72 |
| 95 | R,S | D | c-Pr—NH | H | H | NO₂ | H | CH₃ | 169-172 | 70 |
| 96 | R | D | c-Pr—NH | H | H | NO₂ | H | CH₃ | 83-90 | 75 |
| | | | | | | | | | | +364° |
| | | | | | | | | | | (c = 0.2, |
| | | | | | | | | | | CHCl₃) |
| 97 | R | D | c-Pr—NH | H | CH₃ | NO₂ | H | CH₃ | 155-160 | 79 |
| | | | | | | | | | | +319° |
| | | | | | | | | | | (c = 0.3, |
| | | | | | | | | | | CHCl₃) |
| 98 | R,S | C | 1 | H | H | NO₂ | H | CH₃ | 129-131 | 82 |
| | | | morpholyl | | | | | | | |
| 99 | R,S | C | 1 | H | H | NO₂ | H | CH₃ | 98-101 | 67 |
| | | | piperidyl | | | | | | | |
| 100 | R,S | E | CH₃ONH | H | H | NO₂ | H | CH₃ | 200-201 | 74 |
| 101 | R | E | CH₃ONH | H | H | NO₂ | H | CH₃ | 142-143 | 74 |
| | | | | | | | | | | +317° |
| | | | | | | | | | | (c = 0.2, |
| | | | | | | | | | | CHCl₃) |

### Example 102

### (R,S)-7,8-Dimethoxy-4-methyl-3-methylthiocarbamoyl-1-(4-nitrophenyl)-3H-[2,3]benzodiazepine

A solution of 0.60 g (1.76 mmol) of starting compound I and 1.28 g (17.5 mmol) of methylisocyanate in 30 ml of dichloromethane was heated under reflux for 26 h. The reaction mixture was quenched with water and the two layers were separated. The organic phase was extracted twice with water and after drying evaporated to give a residue that was purified by column chromatography on silica gel using a 2:1 mixture of hexane-ethyl acetate as eluent. Recrystallization from 2-methoxyethanol (4 ml) gave after drying 0.50 g (69%) of the title product with mp.: 221-222° C.

### Examples 103-180

### General Procedures for Reduction of the Nitro Group of the Compounds Obtained in the Above Examples

Method A

2 mmol of the nitro compound were dissolved in a mixture of methanol-dichloromethane and after adding 10 mmol of 98% hydrazine hydrate and 0.5-1.0 g of Raney Nickel catalyst the mixture was stirred at r.t. for 1-24 h. After filtration of the catalyst the filtrate was concentrated and the residue was treated with water and the product was filtered off. Purification of the product was done by recrystallization or column chromatography.

Method B

0.6 g of a Raney Nickel slurry was prehydrogenated in a (2:1) mixture of methanol and dichloromethane. Then 2.0 mmol of nitro compound, dissolved in the above solvent mixture, was added and hydrogenation was carried out at normal pressure. After filtration of the catalyst and evaporation of the solvent the residue was treated as in Method A.

Method C

2.46 g (10.91 mmol) of tin(II) chloride dihydrate was added to a solution of 1.82 mmol of the nitro compound in ethanol and the mixture was stirred and heated to reflux for 3 h. After evaporation of the solvent the residue was triturated with sodium hydrogen carbonate solution and the product was extracted with ethyl acetate. The organic phase was washed with brine, dried and evaporated to give the crude product which was purified by column chromatography.

The amino compounds prepared by the above procedures are shown in the following two Tables:

TABLE 1
Enantiomers of 1-aminophenyl-7,8-dimethoxy-3-(aryl or carbamoyl)
substituted-4,5-dihydro-3H-[2,3]benzodiazepine derivatives

| | | | | | ¹H NMR data |
|---|---|---|---|---|---|
| No. of | | | | Yield (%) | (recorded at 500 MHz in DMSO-d₆ at T = 300K; |
| Example | Structure | Method | Mp (°C.) | [α]_{D} | other solvent is indicated.) |
| 103 | 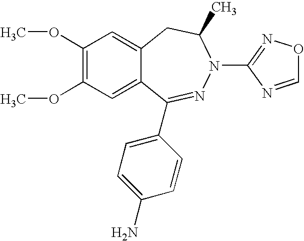 | C | 239-242 | 93[α]_{D}:−657°(c = 0.5,CHCl₃) | (Contains ca. 0.5 molequiv. ofethanol)¹H NMR δ 9.12 (s, 1H), 7.40 (dm, J₁ =8.4 Hz, 2H), 7.07 (s, 1H), 6.61(s, 1H), 6.59 (dm, J₁ = 8.4 Hz,2H), 5.70 (s, 2H), 4.76 (m, 1H),3.84 (s, 3H), 3.63 (s, 3H), 2.92(dd, J₁ = 13.8 Hz, J₂ = 6.2 Hz,1H), 2.52 (dd, J₁ = 13.8 Hz, J₂ =11.7 Hz, 1H), 1.21 (d, J = 6.0 Hz). |
| 104 | 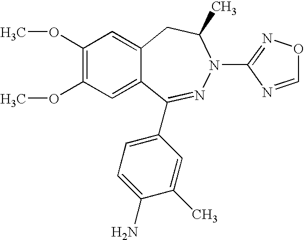 | C | amorphous | 53[α]_{D}:−693°(c = 0.5,CHCl₃) | (Contains ca. 0.5 molequiv. ofchloroform)¹H NMR δ 9.12 (s, 1H), 7.37 (d, J =1.8 Hz, 1H), 7.20 (dd, J₁ = 8.2 Hz,J₂ = 1.8 Hz, 1H), 7.06 (s, 1H),6.62 (d, J = 8.2 Hz, 1H), 6.59 (s,1H), 5.47 (s, 2H), 4.75 (m, 1H),3.83 (s, 3H), 3.62 (s, 3H), 2.92(dd, J₁ = 13.6 Hz, J₂ = 5.9 Hz,1H), 2.50 (1H, overlapping), 2.07(s, 3H), 1.20 (d, J = 5.9 Hz). |
| 105 | 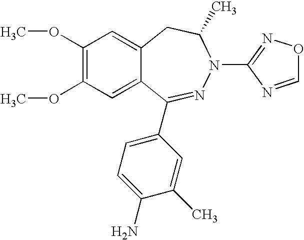 | C | 136-138 | 64 [α]_{D}:+691°(c = 0.3,CHCl₃) | ¹H NMR δ 9.12 (s, 1H), 7.38 (d, J =1.8 Hz, 1H), 7.21 (dd, J₁ = 8.3 Hz,J₂ = 1.8 Hz, 1H), 7.07 (s, 1H),6.62 (d, J = 8.3 Hz, 1H), 6.60 (s,1H), 5.46 (s, 2H), 4.75 (m, 1H),3.84 (s, 3H), 3.62 (s, 3H), 2.91(dd, J₁ = 14.2 Hz, J₂ = 6.2 Hz,1H), 2.51 (dd, J₁ =14.2 Hz, J₂ =11.6 Hz, 1H), 2.08 (s, 3H), 1.20(d, J = 6.2 Hz). |
| 106 | 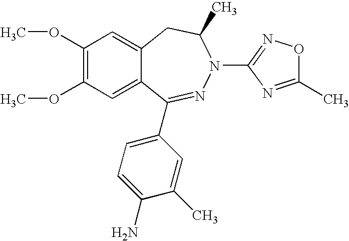 | C | 105-108 | 67[α]_{D}:−574°(c = 0.5,CHCl₃) | ¹H NMR δ 7.36 (d, J = 1.3 Hz, 1H),7.21 (dd, J₁ = 8.3 Hz, J₂ = 1.3 Hz,1H), 7.06 (s, 1H), 6.62 (d, J =8.3 Hz, 1H), 6.58 (s, 1H), 5.45(s, 2H), 4.70 (m, 1H), 3.84 (s,3H), 3.62 (s, 3H), 2.89 (dd, J₁ =13.9 Hz, J₂ = 6.2 Hz, 1H), 2.48(dd, J₁ = 13.9 Hz, J₂ = 11.8 Hz,1H), 1.98 (s, 3H), 1.21 (d, J = 6.2 Hz). |
| 107 | 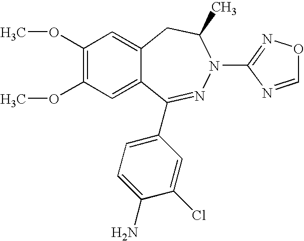 | C | 108-110 | 65[α]_{D}:−628°(c = 0.2,CHCl₃) | ¹H NMR δ 9.14 (s, 1H), 7.61 (d, J =1.8 Hz, 1H), 7.29 (dd, J₁ = 8.5 Hz,J₂ = 1.8 Hz, 1H), 7.08 (s, 1H),6.82 (d, J = 8.5 Hz, 1H), 6.63 (s,1H), 5.95 (s, br, 2H), 4.78 (m,1H), 3.84 (s, 3H), 3.63 (s, 3H),2.94 (dd, J₁ = 13.9 Hz, J₂ = 6.1Hz, 1H), 2.54 (dd, J₁ = 13.9 Hz,J₂ = 11.7 Hz, 1H), 1.21 (d, J = 6.2Hz, 3H). |
| 108 | 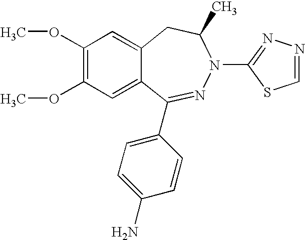 | A | 159-160 | 52[α]_{D}:−416°(c = 0.2,CHCl₃) | ¹H NMR δ 8.79 (s, 1H), 7.37 (dm, J₁ =8.8 Hz, 2H), 7.11 (s, 1H), 6.63(s, 1H), 6.61 (dm, J₁ = 8.8 Hz,2H), 5.74 (s, 2H), 5.05 (m, 1H),3.85 (s, 3H), 3.62 (s, 3H), 3.02(dd, J₁ = 13.9 Hz, J₂ = 4.9 Hz,1H), 2.67 (dd, J₁ = 13.9 Hz, J₂ =10.9 Hz, 1H), 1.23 (d, J = 6.1 Hz). |
| 109 | 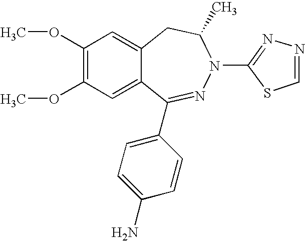 | A | 160-162 | 61[α]_{D}:+419°(c = 0.3,CHCl₃) | ¹H NMR δ 8.79 (s, 1H), 7.37 (dm, J₁ =8.8 Hz, 2H), 7.11 (s, 1H), 6.62(s, 1H), 6.60 (dm, J₁ = 8.8 Hz,2H), 5.74 (s, 2H), 5.04 (m, 1H),3.85 (s, 3H), 3.61 (s, 3H), 3.02(dd, J₁ = 13.9 Hz, J₂ = 4.9 Hz,1H), 2.68 (dd, J₁ = 13.8 Hz, J₂ =10.9 Hz, 1H), 1.22 (d, J = 6.2 Hz). |
| 110 | 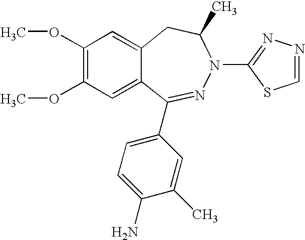 | A | 143 | 85[α]_{D}:−470°(c = 0.5,CHCl₃) | ¹H NMR δ 8.78 (s, 1H), 7.31 (d, J =2.1 Hz, 1H), 7.20 (dd, J₁ = 8.6 Hz,J₂ = 2.1 Hz, 1H), 7.10 (s, 1H),6.64 (d, J = 8.6 Hz, 1H), 6.61 (s,1H), 5.51 (s, 2H), 5.03 (m, 1H),3.85 (s, 3H), 3.60 (s, 3H), 3.01(dd, J₁ =14.1 Hz, J₂ = 5.5 Hz,1H), 2.66 (dd, J₁ = 14.1 Hz, J₂ =10.8 Hz, 1H), 2.09 (s, 3H), 1.22(d, J = 6.1 Hz). |
| 111 | 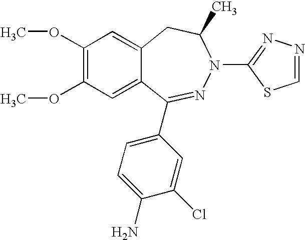 | A | 129-134 | 63[α]_{D}:−255°(c = 0.2,CHCl₃) | ¹H NMR δ 8.81 (s, 1H), 7.51 (d, J =1.5 Hz, 1H), 7.26 (dd, J₁ = 8.5 Hz,J₂ = 1.5 Hz, 1H), 7.11 (s, 1H),6.83 (d, J = 8.5 Hz, 1H), 6.65 (s,1H), 5.96 (br, 2H), 5.09 (m, 1H),3.85 (s, 3H), 3.62 (s, 3H), 3.05(dd, J₁ = 14.0 Hz, J₂ = 4.5 Hz,1H), 2.72 (dd, J₁ = 14.0 Hz, J₂ =10.0 Hz, 1H), 1.07 (d, J = 6.0 Hz). |
| 112 | 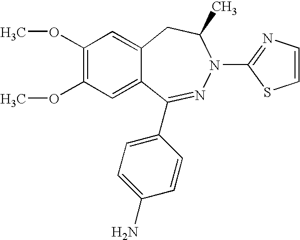 | A | 229-230 | 95[α]_{D}:−486°(c = 0.2,CHCl₃) | ¹H NMR δ 7.40 (dm, J₁ = 8.9 Hz,2H), 7.26 (d, J = 3.7 Hz, 1H),7.08 (s, 1H), 6.81 (d, J = 3.7 Hz,1H), 6.63 (s, 1H), 6.61 (dm, J₁ =8.9 Hz, 2H), 5.69 (s, 2H), 5.05(m, 1H), 3.85 (s, 3H), 3.61 (s,3H), 2.99 (dd, J₁ = 13.9 Hz, J₂ =5.1 Hz, 1H), 2.65 (dd, J₁ = 13.9Hz, J₂ = 10.6 Hz, 1H), 1.18 (d, J =6.0 Hz). |
| 113 | 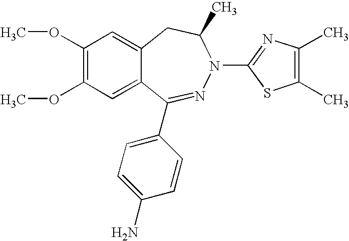 | A | 122-124 | 75[α]_{D}:−558°(c = 0.2,CHCl₃) | ¹H NMR δ 7.36 (dm, J₁ = 7.6 Hz,2H), 7.04 (s, 1H), 6.61 (dm, J₁ =7.6 Hz, 2H), 6.61 (s, 1H), 5.62(s, 2H), 4.99 (m, 1H), 3.83 (s,3H), 3.61 (s, 3H), 2.94 (dd, J₁ =13.7 Hz, J₂ = 4.4 Hz, 1H), 2.62(dd, J₁ = 13.7 Hz, J₂ = 10.5 Hz,1H), 2.14 (s, 3H), 2.05 (s,3H), 1.15 (d, J = 6.1 Hz). |
| 114 | 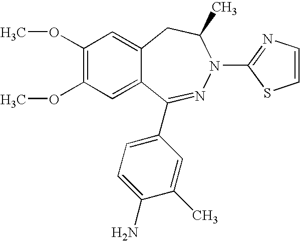 | A | 116-118 | 74[α]_{D}:−527°(c = 0.2,CHCl₃) | ¹H NMR δ 7.34 (d, J = 2.0 Hz, 1H),7.25 (d, J = 3.4 Hz, 1H), 7.22(dd, J₁ = 8.2 Hz, J₂ = 2.0 Hz, 1H),7.07 (s, 1H), 6.81 (d, J = 3.4 Hz,1H), 6.64 (d, J = 8.2 Hz, 1H),6.61 (s, 1H), 5.44 (s, br, 2H),5.04 (m, 1H), 3.84 (s, 3H), 3.60(s, 3H), 2.98 (dd, J₁ = 13.9 Hz,J₂ = 5.3 Hz, 1H), 2.63 (dd, J₁ = 13.9Hz, J₂ = 11.0 Hz, 1H), 2.08 (s,3H), 1.17 (d, J = 6.2 Hz, 3H). |
| 115 | 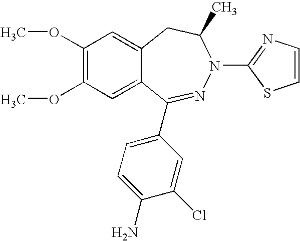 | A | 116-117 | 85[α]_{D}:−281°(c = 0.2,CHCl₃) | ¹H NMR (CDCl₃) δ 7.66 (d, J = 2.0Hz, 1H), 7.43 (dd, J₁ = 8.3 Hz,J₂ =2.0 Hz, 1H), 7.31 (d, J = 3.6Hz, 1H), 6.81 (s, 1H), 6.77 (d,J = 8.3 Hz, 1H), 6.69 (s, 1H), 6.65(d, J = 3.6 Hz, 1H), 5.35 (m,1H), 4.32 (br, 2H), 3.95 (s, 3H),3.73 (s, 3H), 3.08 (dd, J₁ = 14.1Hz, J₂ = 4.5 Hz, 1H), 2.82 (dd,J₁ = 14.1 Hz, J₂ = 8.6 Hz, 1H), 1.28(d, J = 6.4 Hz, 3H). |
| 116 | 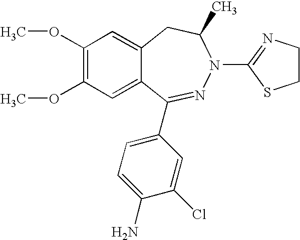 | A | 232-234 | 78[α]_{D}:−170°(c = 0.2,CHCl₃) | ¹H NMR δ 7.30 (d, J = 8.2 Hz, 2H),7.01 (s, 1H), 6.60 (s, 1H), 6.57(d, J = 8.2 Hz, 2H), 5.56 (s, 2H),4.89 (m, 1H), 4.05 (m, 1H), 3.93(m, 1H), 3.83 (s, 3H), 3.61 (s,3H), 3.13 (m, 1H), 3.06 (m, 1H),2.92 (dd, J₁ = 14.2 Hz, J₂ = 5.1Hz, 1H), 2.60 (dd, J₁ = 14.2 Hz,J₂ = 9.5 Hz, 1H), 1.10 (d, J = 6.2 Hz). |
| 117 | 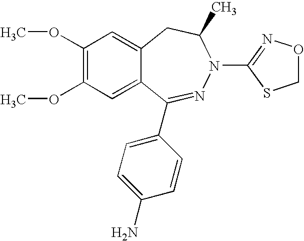 | C | Foam(ca. 92- 95) | 58[α]_{D}:−232°(c = 0.2,CHCl₃) | ¹H NMR δ 7.28 (dm, J₁ = 8.4 Hz,2H), 7.04 (s, 1H), 6.60 (s, 1H),6.57 (dm, J₁ = 8.4 Hz, 2H), 5.66(s, 2H), 5.48 (d, J = 6.2 Hz, 1H),5.46 (d, J = 6.2 Hz, 1H), 4.63 (m,1H), 3.84 (s, 3H), 3.62 (s, 3H),2.93 (dd, J₁ = 13.9 Hz, J₂ = 5.0Hz, 1H), 2.57 (dd, J₁ = 13.9 Hz,J₂ = 10.3 Hz, 1H), 1.18 (d, J = 6.2 Hz). |
| 118 | 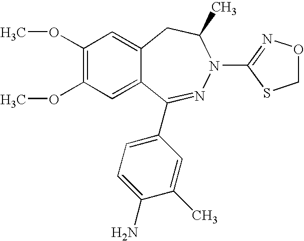 | C | 194-197 | 71[α]_{D}:−327°(c = 0.2,CHCl₃) | ¹H NMR δ 7.24 (d, J = 2.0 Hz, 1H),7.12 (dd, J₁ = 8.7 Hz, J₂ = 2.0 Hz,1H), 7.04 (s, 1H), 6.61 (d, J =8.7 Hz, 1H), 6.60 (s, 1H), 5.48(d, J = 6.0 Hz, 1H), 5.46 (d, J =6.0 Hz, 1H), 5.43 (s, 2H), 4.62(m, 1H), 3.84 (s, 3H), 3.61 (s,3H), 2.92 (dd, J₁ = 14.1 Hz, J₂ =5.3 Hz, 1H), 2.55 (dd, J₁ = J₂ =14.1 Hz, 1H), 2.06 (s, 3H), 1.19(d, J = 6.4 Hz). |
| 119 | 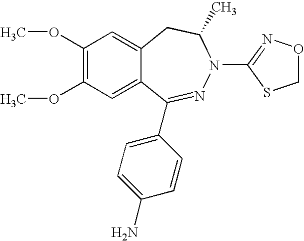 | C | 134-135 | 61[α]_{D}:−224°(c = 0.2,CHCl₃) | ¹H NMR δ 7.29 (dm, J₁ = 8.6 Hz,2H), 7.04 (s, 1H), 6.60 (s, 1H),6.57 (dm, J₁ = 8.6 Hz, 2H), 5.65(s, 2H), 5.48 (d, J = 6.1 Hz, 1H),5.46 (d, J = 6.1 Hz, 1H), 5.63 (m,1H), 3.84 (s, 3H), 3.62 (s, 3H),2.94 (dd, J₁ = 13.9 Hz, J₂ = 5.0Hz, 1H), 2.57 (dd, J₁ = 13.9 Hz,J₂ = 9.9 Hz, 1H), 1.18 (d, J = 6.1 Hz). |
| 120 | 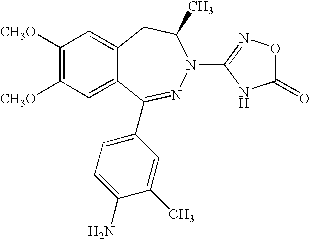 | B | Foam(ca. 159-164) | 62[α]_{D}:−323°(c = 0.2,CHCl₃) | (Contains ca. 0.3 molequiv. ofethyl acetate)¹H NMR δ 11.75 (s, br, 1H), 7.51(d, J = 2.0 Hz, 1H), 7.25 (dd, J₁ =8.5 Hz, J₂ = 2.0 Hz, 1H), 7.05 (s,1H), 6.59 (d, J₁ = 8.5 Hz, 1H),6.55 (s, 1H), 5.50 (s, br, 2H),4.47 (m, 1H), 3.84 (s, 3H), 3.62(s, 3H), 2.91 (dd, J₁ = 13.9 Hz,J₂ = 5.9 Hz, 1H), 2.49 (dd,overlapping, 1H), 1.22 (d, J = 6.0 Hz). |
| 121 | 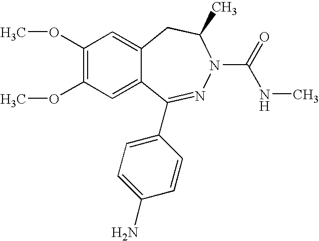 | A,B,C | 140-144withtrans-formationat 108) | 77 (A)[α]_{D}:−255°(c = 0.5,CHCl₃) | ¹H NMR δ 7.44 (dm, J₁ = 8.6 Hz,2H), 7.01 (s, 1H), 6.57 (dm, J₁ =8.6 Hz, 2H), 6.55 (s, 1H), 6.15(q, J = 4.8 Hz, 1H), 5.63 (s,2H), 4.84 (m, 1H), 3.83 (s, 3H),3.62 (s, 3H), 2.79 (dd, J₁ = 13.8Hz, J₂ = 5.8 Hz, 1H), 2.62 (d, J =4.8 Hz, 3H), 2.41 (dd, J₁ = 13.8Hz, J₂ = 11.7 Hz, 1H), 1.07 (d, J =6.4 Hz). |
| 122 | 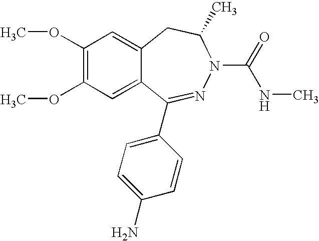 | A | 130-134 | 75[α]_{D}:+206°(c = 0.2,CHCl₃) | ¹H NMR δ 7.44 (dm, J₁ = 8.6 Hz,2H), 7.01 (s, 1H), 6.57 (dm, J₁ =8.6 Hz, 2H), 6.54 (s, 1H), 6.15(q, J = 4.9 Hz, 1H), 5.63 (s, 2H),4.83 (m, 1H), 3.83 (s, 3H), 3.61(s, 3H), 2.78 (dd, J₁ = 13.9 Hz, J₂ =5.9 Hz, 1H), 2.41 (dd, J₁ = J₂ =13.9 Hz, 1H), 1.07 (d, J = 6.2 Hz). |
| 123 | 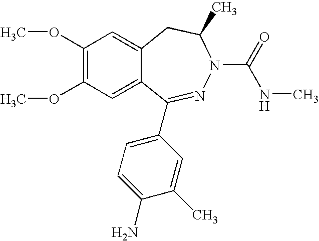 | A | 147 | 84[α]_{D}:−285°(c = 0.5,CHCl₃) | ¹H NMR δ 7.37 (d, J = 1.8 Hz, 1H),7.25 (dd, J₁ = 8.2 Hz, J₂ = 1.8 Hz,1H), 7.00 (s, 1H), 6.60 (d, J =8.2 Hz, 1H), 6.53 (s, 1H), 6.13(q, J = 4.9 Hz, 1H), 5.39 (s, 2H),4.83 (m, 1H), 3.82 (s, 3H), 3.60(s, 3H), 2.79 (dd, J₁ = 13.9 Hz, J₂ =6.1 Hz, 1H), 2.61 (d, J = 4.9Hz, 1H), 2.39 (dd, J₁ = 13.9 Hz, J₂ =11.9 Hz, 1H), 2.07 (s, 3H), 1.06(d, J = 5.9 Hz). |
| 124 | 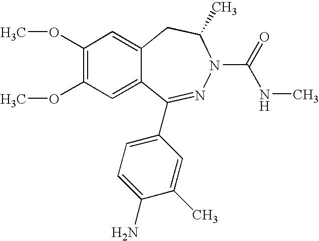 | A | 157-159 | 78[α]_{D}:+308°(c = 0.2,CHCl₃) | ¹H NMR δ 7.41 (d, J = 1.7 Hz, 1H),7.26 (dd, J₁ = 8.7 Hz, J₂ = 1.7 Hz,1H), 7.01 (s, 1H), 6.60 (d, J =8.7 Hz, 1H), 6.54 (s, 1H), 6.13(d, J = 4.2 Hz, 1H), 5,39 (s, 2H),4.84 (m, 1H), 3.83 (s, 3H), 3.61(s, 3H), 2.79 (dd, J₁ = 13.7 Hz, J₂ =5.4 Hz, 1H), 2.62 (d, J = 4.3Hz, 3H), 2.40 (dd, J₁ = J₂ = 13.7Hz, 1H), 2.08 (s, 3H), 1.07 (d, J = 6.4 Hz). |
| 125 | 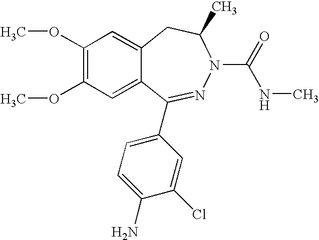 | A | 119-12 | 82[α]_{D}:−125°(c = 0.2,CHCl₃) | ¹H NMR δ 7.72 (d, J = 2.0 Hz, 1H),7.32 (dd, J₁ = 8.4 Hz, J₂ = 2.0Hz, 1H), 7.02 (s, 1H), 6.79 (d, J =8.4 Hz, 1H), 6.56 (s, 1H), 6.32(q, J = 4.7 Hz, 1H), 5.87 (s, 2H),4.88 (m, 1H), 3.83 (s, 3H), 3.62(s, 3H), 2.81 (dd, J₁ = 14.0 Hz, J₂ =6.0 Hz, 1H), 2.43 (dd, J₁ = 14.0, J =11.9 Hz, 1H), 1.07 (d, J = 6.2 Hz). |
| 126 | 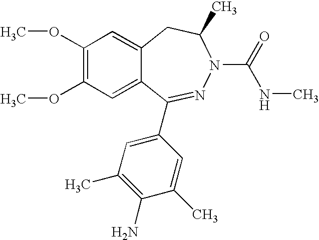 | A | 110-113 | 85[α]_{D}:−362°(c = 0.2,CHCl₃) | ¹H NMR δ 7.24 (s, 2H), 7.00 (s,1H), 6.54 (s, 1H), 6.13 (q, J =4.2 Hz, 1H), 5.09 (s, 2H), 4.83(m, 1H), 3.83 (s, 3H), 3.60 (s,3H), 2.79 (dd, J₁ = 13.8 Hz, J₂ =5.6 Hz, 1H), 2.62 (d, J = 4.8 Hz,3H), 2.38 (dd, J₁ = J₂ = 13.8 Hz,1H), 2.10 (s, 6H), 1.07 (d, J =5.9 Hz). |
| 127 | 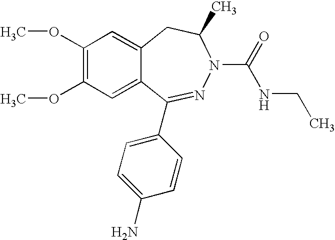 | A | 122-124 | 84[α]_{D}:−266 °(c = 0.2,CHCl₃) | ¹H NMR δ 7.42 (dm, J₁ = 8.7 Hz,2H), 7.01 (s, 1H), 6.56 (dm, J₁ =8.7 Hz, 2H), 6.55 (s, 1H), 6.25(t, J = 4.9 Hz, 1H), 5.64 (s, 2H),4.83 (m, 1H), 3.82 (s, 3H), 3.61(s, 3H), 3.08 (m, 2H), 2.78 (dd, J₁ =13.9 Hz, J₂ = 5.9 Hz, 1H), 2.41(dd, J₁ = J₂ = 13.9 Hz, 1H), 1.06(d, J = 6.4 Hz), 1.01 (t, J = 7.2 Hz) |
| 128 | 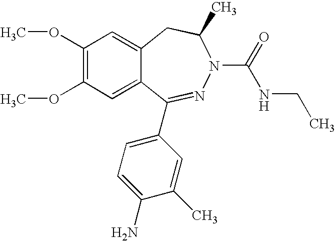 | A | 107-110 | 65[α]_{D}:−300°(c = 0.2,CHCl₃) | ¹H NMR δ 7.36 (s, 1H), 7.26 (d, J =8.1 HZ, 1H), 7.01 (s, 1H), 6.61(d, J = 8.1 Hz, 1H), 6.55 (s, 1H),6.22 (t, J = 5.2 Hz), 5.39 (s,2H), 4.84 (m, 1H), 3.83 (s, 3H),3.61 (s, 3H), 3.09 (m, 2H), 2.80(dd, J₁ = 13.0 Hz, J₂ = 5.3 Hz,1H), 2.41 (dd, J₁ = J₂ = 13.0 Hz,1H), 2.08 (s, 3H), 1.07 (d, J =6.0 Hz), 1.02 (t, J = 7.1 Hz). |
| 129 | 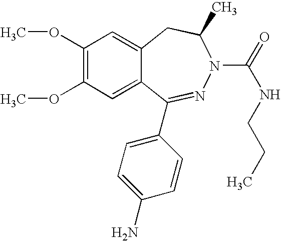 | A | 101-104 | 73[α]_{D}:−203°(c = 0.2,CHCl₃) | ¹H NMR δ 7.42 (dm, J₁ = 8.0 Hz,2H), 7.01 (s, 1H), 6.58 (dm, J₁ =8.0 Hz, 2H), 6.56 (s, 1H), 6.25(t, J = 5.8 Hz, 1H), 5.63 (s, br,2H), 4.85 (m, 1H), 3.83 (s, 3H),3.62 (s, 3H), 3.03 (m, 2H), 2.80(dd, J₁ = 13.7 Hz, J₂ = 5.0 Hz,1H), 2.43 (dd, J₁ = 13.7 Hz, J₂ =11.7 Hz, 1H), 1.43 (m, 2H), 1.07(d, J = 6.0 Hz), 0.82 (t, J = 7.6Hz). |
| 130 | 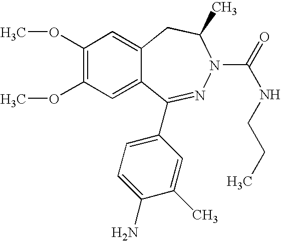 | A | 92-95 | 71[α]_{D}:−237°(c = 0.2,CHCl₃) | ¹H NMR δ 7.34 (d, J = 2.1 Hz, 1H),7.24 (dd, J₁ = 8.6 Hz, J₂ = 2.1 Hz,1H), 7.00 (s, 1H), 6.61 (d, J =8.6 Hz, 1H), 6.55 (s, 1H), 6.22(t, J = 6.4 Hz, 1H), 5.39 (s, br,2H), 4.83 (m, 1H), 3.82 (s, 3H),3.60 (s, 3H), 3.02 (m, 2H), 2.80(dd, J₁ = 13.7 Hz, J₂ = 5.9 Hz,1H), 2.41 (dd, J₁ = 13.7 Hz, J₂ =11.7 Hz, 1H), 2.07 (s, 3H), 1.43(m, 2H), 1.06 (d, J = 6.0 Hz, 3H),0.82 (t, J = 7.3 Hz, 3H). |
| 131 | 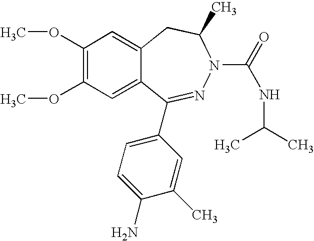 | A | 115-117 | 74[α]_{D}:−226°(c = 0.2,CHCl₃) | ¹H NMR δ 7.26 (d, J = 2.0 Hz, 1H),7.20 (dd, J₁ = 8.3 Hz, J₂ = 2.0 Hz,1H), 7.00 (s, 1H), 6.63 (d, J =8.3 Hz, 1H), 6.58 (s, 1H), 5.88(d, J = 7.7 Hz, 1H), 5.42 (s, br,2H), 4.85 (m, 1H), 3.82 (s, 3H),3.73 (m, 1H), 3.60 (s, 3H), 2.81(dd, J₁ = 13.5 Hz, J₂ = 5.3 Hz,1H), 2.43 (dd, J₁ = 13.5 Hz, J₂ =11.7 Hz, 1H), 2.07 (s, 3H), 1.14(d, J = 6.4 Hz, 3H), 1.06 (d, J =6.4 Hz, 6H). |
| 132 | 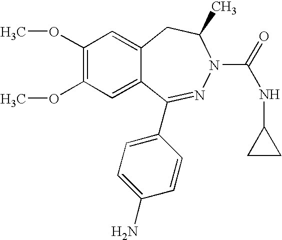 | A | 116-119 | 80[α]_{D}:−222°(c = 0.2,CHCl₃) | ¹H NMR δ 7.38 (dm, J₁ = 8.3 Hz,2H), 7.01 (s, 1H), 6.57 (dm, J₁ =8.3 Hz, 2H), 6.57 (s, 1H), 6.14(d, J = 2.2 Hz, 1H), 5.64 (s, br,2H), 4.85 (m, 1H), 3.83 (s, 3H),3.62 (s, 3H), 2.80 (dd, J₁ = 13.6Hz, J₂ = 5.4 Hz, 1H), 2.50 (m,overlapping), 2.42 (dd, J₁ = 13.6Hz, J₂ = 11.7 Hz, 1H), 1.08 (d, J =6.2 Hz), 0.6-0.4 (m, 4H). |
| 133 | 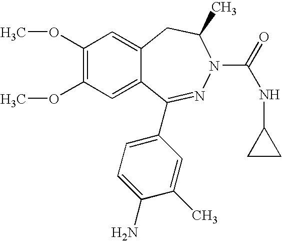 | A | 112-114 | 78[α]_{D}:−260°(c = 0.2,CHCl₃) | ¹H NMR δ 7.32 (d, J = 1.8 Hz, 1H),7.22 (dd, J₁ = 8.3 Hz, J₂ = 1.8 Hz,1H), 7.01 (s, 1H), 6.61 (d, J =8.3 Hz, 1H), 6.56 (s, 1H), 6.13(d, J = 2.9 Hz, 1H), 5.40 (s, br,2H), 4.84 (m, 1H), 3.83 (s, 3H),3.61 (s, 3H), 2.80 (dd, J₁ = 13.6Hz, J₂ = 5.6 Hz, 1H), 2.50 (m,overlapping), 2.40 (dd, J₁ = 13.6Hz, J₂ = 11.6 Hz, 1H), 2.07 (s,3H), 1.08 (d, J = 6.0 Hz, 3H),0.6-0.4 (m, 4H). |
| 134 | 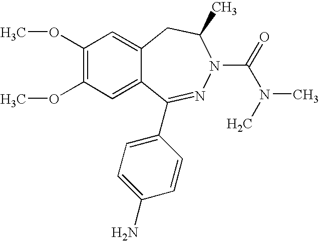 | A | 258-260 | 80[α]_{D}:−434°(c = 0.2,CHCl₃) | ¹H NMR δ 7.36 (dm, J₁ = 8.2 Hz,2H), 7.00 (s, 1H), 6.57 (s, 1H),6.56 (dm, J₁ = 8.2 Hz, 2H), 5.67(s, 2H), 4.46 (m, 1H), 3.82 (s,3H), 3.63 (s, 3H), 2.76 (dd, J₁ =12.9 Hz, J₂ = 5.6 Hz, 1H), 2.35(dd, J₁ = J₂ = 12.9 Hz, 1H), 1.16(d, J = 4.8 Hz) |
| 135 | 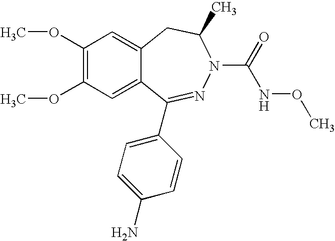 | C | 143-145 | 26[α]_{D}:−260°(c = 0.1,CHCl₃) | (Contains ca. 0-4 molequiv. ofethyl acetate)¹H NMR δ 9.21 (s, 1H), 7.47 (dm,J₁ = 9.0 Hz, 2H), 7.02 (s, 1H), 6.56(s, 1H), 6.55 (dm, J₁ = 9.0 Hz,2H), 5.57 (s, 2H), 4.76 (m, 1H),3.83 (s, 3H), 3.62 (s, 3H), 3.53(s, 3H), 2.80 (dd, J₁ = 13.0 Hz,J₂ = 5.3 Hz, 1H), 2.43 (dd, J₁ = J₂ =13.0 Hz, 1H), 1.12 (d, J = 6.5 Hz). |

TABLE 2
Racemic 1-aminophenyl-7,8-dialkoxy-3-
(aryl or carbamoyl) substituted-4,5-dihydro-3H-
[2,3]benzodiazepine derivatives

| No. of | | | | |
|---|---|---|---|---|
| Example | Structure | Method | Mp (° C.) | Yield (%) |
| 136 | 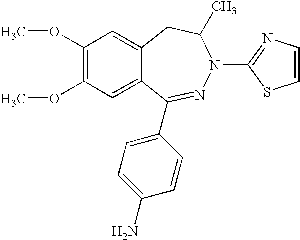 | A | 119-122 | 69 |
| 137 | 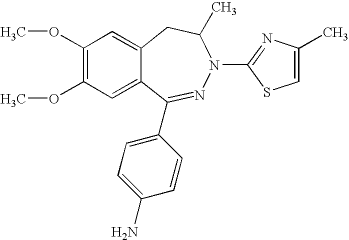 | A | 122-124 | 88 |
| 138 | 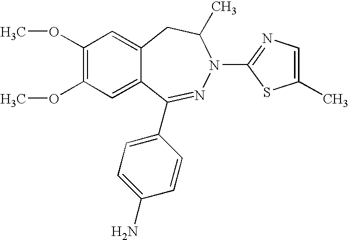 | A | 120-122 | 74 |
| 139 | 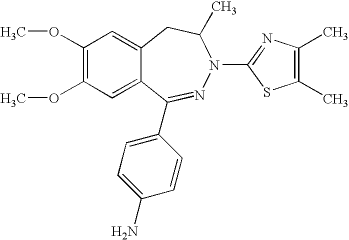 | A | 121-124 | 87 |
| 140 | 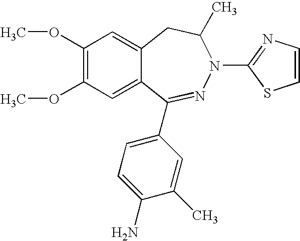 | A | 109-113 | 76 |
| 141 | 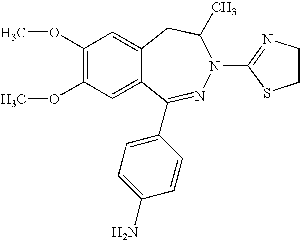 | A | 121-123 | 84 |
| 142 | 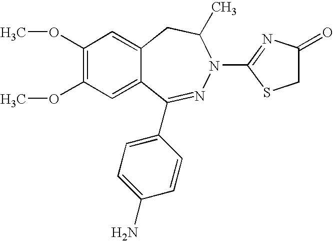 | A | 166-168 | 94 |
| 143 | 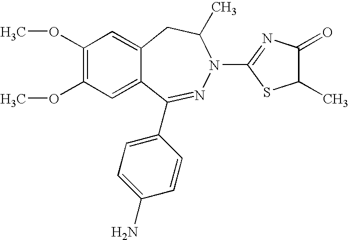 | A | 184-188 | 77 |
| 144 | 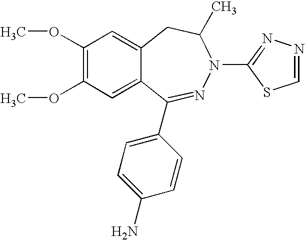 | A | 152-153 | 71 |
| 145 | 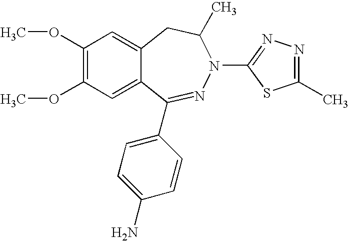 | A | 220-222 | 73 |
| 146 | 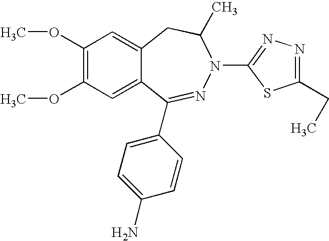 | A | 208-209 | 76 |
| 147 | 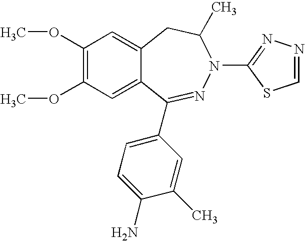 | A | 108-110 | 74 |
| 148 | 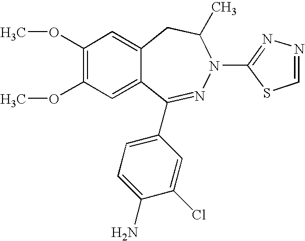 | A | 125-130 | 69 |
| 149 | 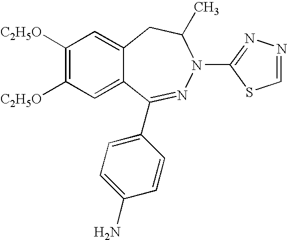 | A | 108-113 | 78 |
| 150 | 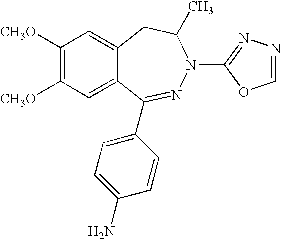 | A | 100-105 | 74 |
| 151 | 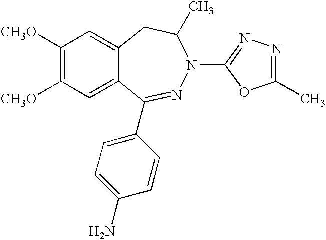 | A | 209-211 | 72 |
| 152 | 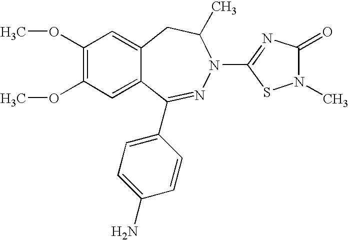 | A | 260-262 | 86 |
| 153 | 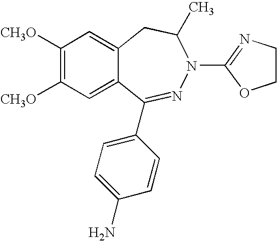 | B | 135-137 | 80 |
| 154 | 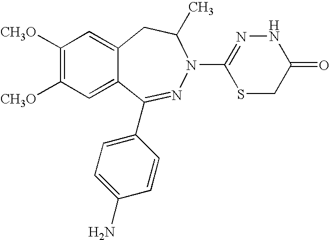 | A | 159-163 | 64 |
| 155 | 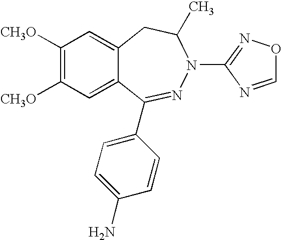 | C | 227-229 | 61 |
| 156 | 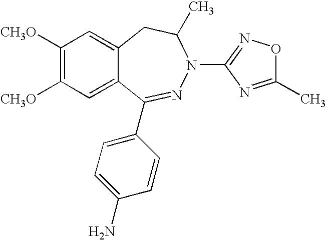 | C | 233-236 | 78 |
| 157 | 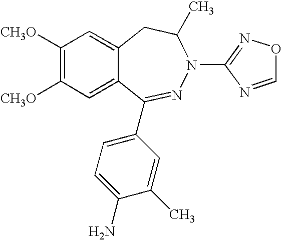 | C | 234-237 | 58 |
| 158 | 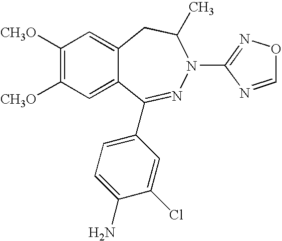 | C | 232-235 | 56 |
| 159 | 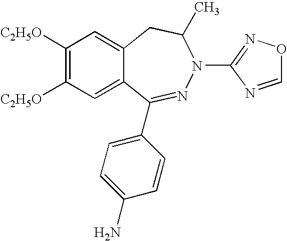 | C | 158-162 | 56 |
| 160 | 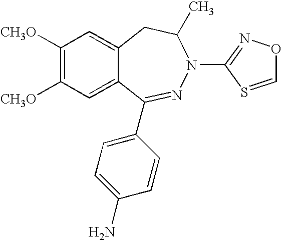 | C | 117-119 | 59 |
| 161 | 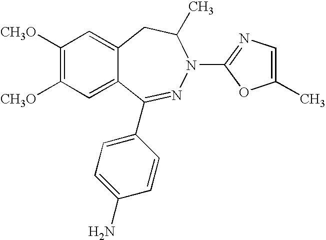 | A | 103-107 | 80 |
| 162 | 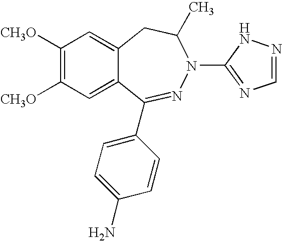 | A | 152-155 | 73 |
| 163 | 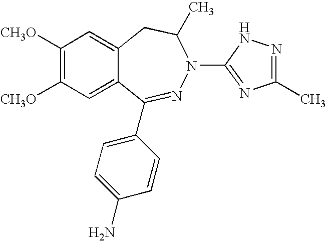 | A | 164-167 | 87 |
| 164 | 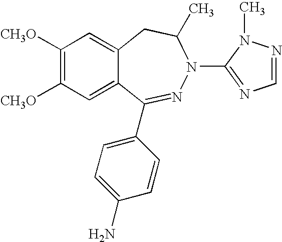 | A | 160-162 | 72 |
| 165 | 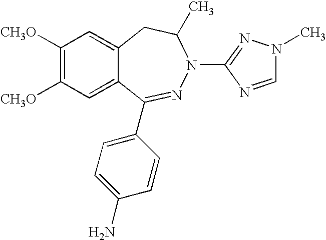 | A | 203-205 | 78 |
| 166 | 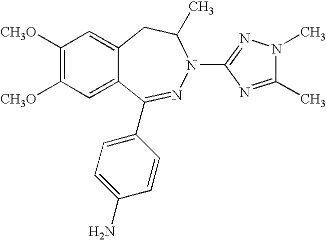 | A | 243-246 | 56 |
| 167 | 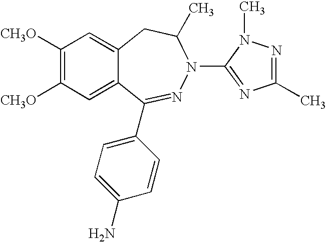 | A | 113-115 | 80 |
| 168 | 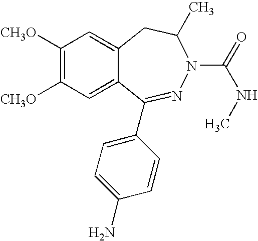 | A | 105-107 | 73 |
| 169 | 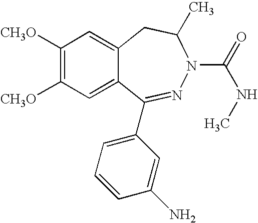 | A | 103-108 | 67 |
| 170 | 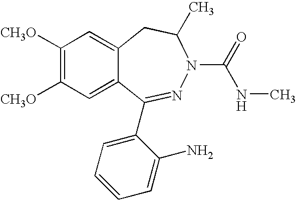 | A | 88-91 | 74 |
| 171 | 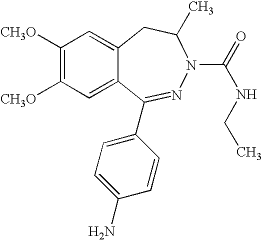 | A | 169-170 | 71 |
| 172 | 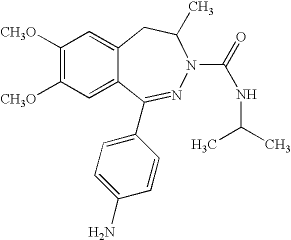 | A | 109-111 | 85 |
| 173 | 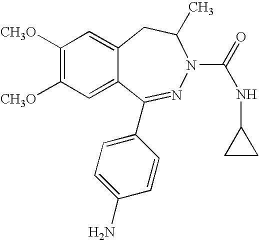 | A | 120-123 | 65 |
| 174 | 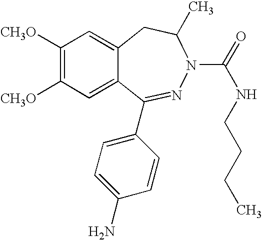 | A | 204-207 | 94 |
| 175 | 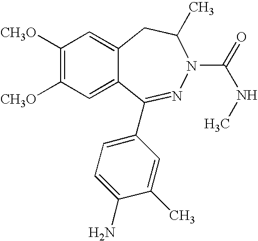 | A | 119-122 | 92 |
| 176 | 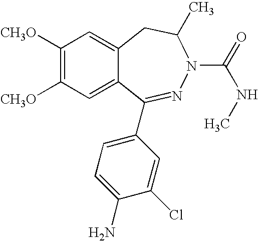 | A | 119-120 | 78 |
| 177 | 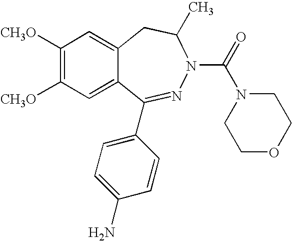 | A | 124-127 | 62 |
| 178 | 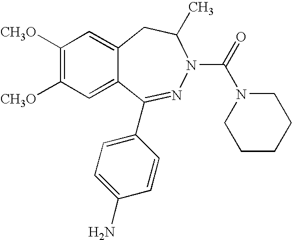 | A | 129-132 | 67 |
| 179 | 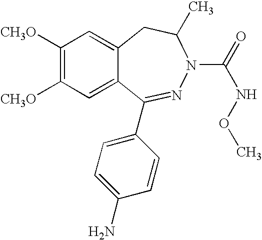 | C | 173-174 | 36 |
| 180 | 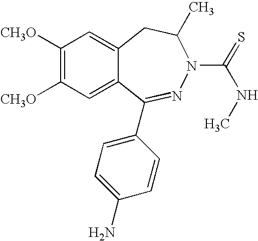 | C | 235-237 | 70 |

### Examples 181-191

### General Procedure for Synthesis of 2,3-Benzodiazepines Containing 1-Acylamino-Phenyl Group

To a stirred solution of a 2,3-benzodiazepine containing an aminophenyl substituent from one of the previous examples, in dichloromethane, an excess of acetic anhydride (or in case of Example 191 methyl isocyanate) was added and the reaction mixture was kept at r.t. After completion of the reaction, the mixture was washed with sodium hydrogen carbonate solution and water, then dried and evaporated to dryness.

The following compounds were prepared by this procedure:

| | | Configur- | | |
|---|---|---|---|---|
| No. of | | ation | | Yield (%) |
| Example | Structure | at C-4 | Mp (° C.) | [α]_{D} |
| 181 | 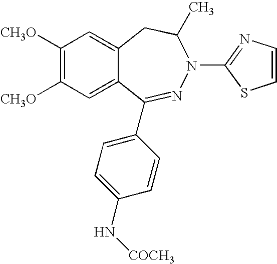 | R, S | 130-134 | 54 |
| 182 | 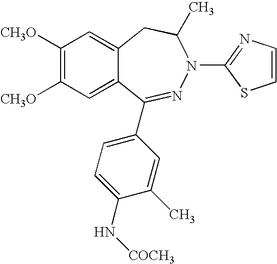 | R, S | 209-211 | 77 |
| 183 | 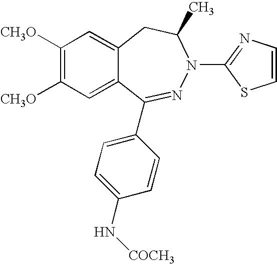 | R | 156-158 | 90−50°(c = 0.2CHCl₃) |
| 184 | 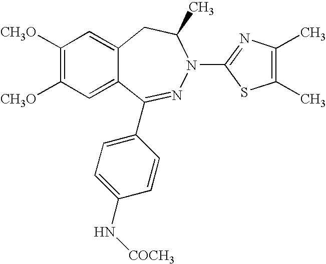 | R | 238-239 | 54−143°(c = 0.2CHCl₃) |
| 185 | 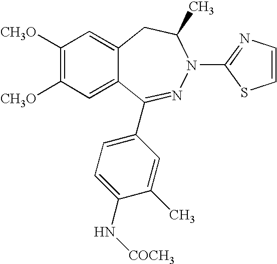 | R | 229-231 | 62−64°(c = 0.2CHCl₃) |
| 186 | 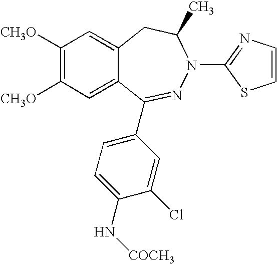 | R | 213-215 | 65−82°(c = 0.2CHCl₃) |
| 187 | 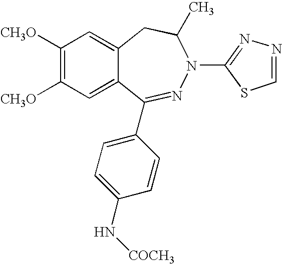 | R, S | 150-151 | 53 |
| 188 | 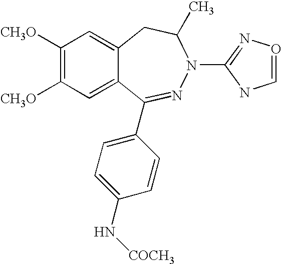 | R, S | 207-210 | 87 |
| 189 | 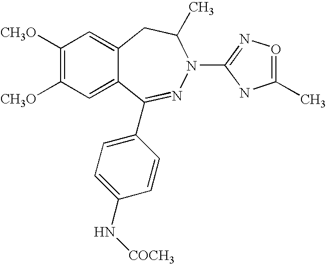 | R, S | 152-154 | 80 |
| 190 | 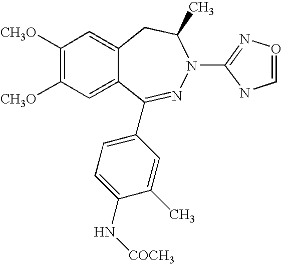 | R | 234-236 | 75−621°(c = 0.2CHCl₃) |
| 191 | 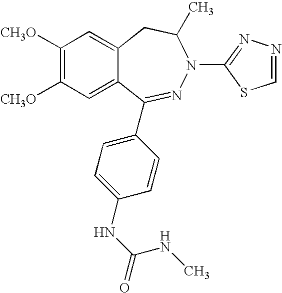 | R, S | 160-162 | 67 |

### BIOLOGICAL EXAMPLES

The biological activity of the compounds of formula (I) are exemplified by the following methods.

In Vitro Tests

The new 2,3-benzodiazepine atypical antipsychotic agents of formula (I) of the present invention influence dopaminergic neurotransmission by a unique indirect fashion, differently from both the first and second generation antipsychotics. The in vitro binding profile of these compounds (Table 3) differs from that of the first generation antipsychotics which bind to dopaminergic receptors and second generation antipsychotics, representatives of which show affinity for dopaminergic, serotonergic, or adrenergic receptors.

TABLE 3
The in vitro binding profile of some compounds
of invention and clozapine to central dopaminergic,
serotonergic and adrenergic receptors

| Com- | | | | | | | | |
|---|---|---|---|---|---|---|---|---|
| pounds | | | | | | | | |
| (Number | | | | | | | | |
| of | | | | | | | | |
| Example) | D₁ | D₂ | D₃ | D₄ | 5HT_{1A} | 5HT_{2A} | α₁ | α₂ |
| 121 | >10⁴ | >10⁴ | >10⁴ | >10⁴ | >10⁴ | >10⁴ | >10⁴ | >10⁴ |
| 123 | >10⁴ | >10⁴ | >10⁴ | >10⁴ | >10⁴ | >10⁴ | >10⁴ | >10⁴ |
| 104 | >10⁴ | >10⁴ | >10⁴ | >10⁴ | >10⁴ | >10⁴ | >10⁴ | >10⁴ |
| 110 | >10⁴ | >10⁴ | >10⁴ | >10⁴ | >10⁴ | >10⁴ | >10⁴ | >10⁴ |
| Cloza- | 290 | 130 | 240 | 47 | 140 | 8.9 | 4 | 33 |
| pine* | | | | | | | | |

Data are expressed in nM
*Miyamoto, S., Therapeutics of Scizophrenia. In: Davis, K. L., Charney, D., Coyle, J. T. et al, eds. Neuropsychopharmacology: The 5th Generation of Progress. Philadelphia, Pa: Lippincott Williams & Wilkins, 2002, p 778.
D₂ receptor binding was determined using rat striatal membranes (ligand: [³H]sulpirid, D₁ and D₂ binding in human recombinant CHO cells (ligands: [³H]SCH-23390 and [³H]spiperone, resp.), D₄ binding in human recombinant CHO-K1 cells (ligand: [³H]spiperone), 5HT_{1A} in rat hippocampal membranes (ligand: [³H]-8-OH-DPAT, 5HT_{2A} in rat cerebral cortical membranes (ligand: [³H]ketanserin, α₁ and α₂ binding in rat cerebral cortical membranes (ligands: [³H]prazosin and [³H]yohimbine, resp.).

Acute administration of atypical antipsychotics results in enhanced accumulation of dihydroxyphenylalanine (DOPA) in the eminentia mediana in rats after the inhibition of DOPA decarboxylase. In contrast, DOPA accumulation is not altered by the typical antipsychotics (Andersson, G, Albinsson, A, Pettersson, G, Arzneimittelforschung. 1990, 40, 237; Gudelsky, G. A., Meltzer, H. Y. Neuropsychopharmacology, 1989, 2, 45).

The effect of the compound of Example 121 on DOPA-accumulation was measured by analyzing the levels of DOPA in eminentia mediana. Sprague-Dawley (240-280 g, male) rats were treated (ip) with compound of Example 121 or with reference compounds (chlorpromazine, clozapine) at doses indicated in Table 4. Thirty min after the treatments they received NSD-1015 (100 mg/kg), a DOPA-decarboxylase inhibitor, and the animals were sacrificed 30 min later. Eminentia mediana (EM), were dissected out from the brains on ice-cold plates and kept at −70° C. until they were assayed for DOPA by HPL-EC. Statistical analysis was carried out using one-way ANOVA followed by Duncan test.

TABLE 4
Effect of compound of Example
121 on DOPA accumulation in EM

| Treatment | | |
|---|---|---|
| & dose ip. (mg/kg) | | DOPA levels |
| (Referents & | DOPA levels | (pg/tissue |
| compound of Example | (pmol/mg prot) | block) |
| Saline | 49.9 ± 5.4 | 422 ± 30 |
| CPZ.; 10 | 56.4 ± 4.1 | n.t. |
| CLOZ; 20 | 87.4 ± 9.5** | n.t. |
| 121; 10 | n.t. | 484 ± 34 |
| 121; 20 | n.t. | 652 ± 69* |
| 121; 40 | 94.0 ± 11.4** | n.t. |

CPZ = Chlorpromazine;
CLOZ = Clozapine
Summary data of two separate experiments. Bars represent the group means ± SEM. Animals in the CPZ-group were treated with NSD-1015 90 min after the CPZ treatment. Statistically different (*p < 0.05; **p < 0.01) from the saline group.
n.t. = not tested

As Table 4 shows, the compound of Example 121 and the atypical antipsychotic clozapine significantly increased the levels of DOPA in the EM of rats. Chlorpromazine, a typical antipsychotic had no effect.

The unique spectrum of in vitro activity of new atypical 2,3-benzodiazepines of formula (I) is presented in Table 5.

TABLE 5
In vitro efficacy of new atypical
2,3-benzodiazepines of formula (I)

Inhibition in % (conc: 10 μM)

| Compounds of | Adenosine | Melatonin (MT2) |
|---|---|---|
| Example | transporter | receptor |
| 121 | 87 | 80 |
| 123 | 90 | 64 |
| 104 | 96 | 80 |
| 110 | 94 | 95 |

It is well known from the literature that both adenosine and melatonin influence dopaminergic neurotransmission. (Weiss, S. M. et al, Neurology, 2003, 61, S88-S93; Miyamoto, S., Therapeutics of Scizophrenia. In: Davis, K. L., Charney, D., Coyle, J. T. et al, eds. Neuropsychopharmacology: The 5th Generation of Progress. Philadelphia, Pa.: Lippincott Williams & Wilkins, 2002, pp 775-807; Zisapel, N. Cell. Mol. Neurobiol. 2001, 21, 605). Thus, the atypical antipsychotic efficacy of the compounds of formula (I) may be related to the activity on the adenosine transporter and melatonin receptor.

The compounds prepared according to examples 121, 123, 104, 110, 112, and 183 were also tested in an in vitro “spreading depression” model to determine the AMPA antagonistic effect of the compounds of formula (I). Specifically, the inhibition of AMPA induced “spreading depression” caused by glutamate agonists (i.e., AMPA or kainate) was studied in isolated chicken retina. By way of background, the “spreading depression” model has shown that AMPA antagonists prolong the latency of the development of the “spreading depression” caused by AMPA (5 μM).

The compounds of the present invention inhibited the AMPA-induced “spreading depression” with an IC₅₀ value of greater than 20 μM. When these results were compared to the results shown for AMPA antagonists in Table 1 of U.S. Pat. No. 6,858,605, it was shown that the compounds of the present invention do not exhibit AMPA antagonism.

In Vivo Tests

The in vivo antipsychotic effect of the compounds is exemplified in the following experiments. Test substances were suspended in 2% Tween-80.

Anti-apomorphine effects were investigated in the climbing test in mice mediated through the mesolimbic and stereotypy test in rats mediated through the nigrostriatal system. Conventional antipsychotics antagonize both apomorphine-induced behaviours, while known atypical antipsychotics are weaker against apomorphine stereotypy.

The climbing test was carried out according to Protais et al. (Protais, P. et al. Psychopharmacologia, 1976, 50, 1.) Stereotyped behavior was induced in food deprived (for 16 hours) male CD1 mice weighing 20-25 g body by apomorphine HCl (SIGMA) in a s.c. dose of 2 mg/kg. Mice were placed individually into cylinders having 12 cm diameter and consisting of vertical bars of 2 mm diameter where apomorphine treated animals tended to adopt a vertical position in contrary to vehicle treated controls. Test substances were applied ip. 30 min before apomorphine.

10 and 20 min after apomorphine treatment the climbing behaviour was evaluated by scores of 0-2. 10 mice/group were used. The scores of the two readings were summed individually, meaned and compared to the control. The ED₅₀ values were calculated by Litchfield-Wilcoxon's method (Litchfield Jr. J. T., Wilcoxon, F. J. Pharmacol. Exp. Ther. 1949, 96, 49,).

Apomorphine-induced stereotypy was investigated according to Costall and Naylor (B. Costall and R. J. Naylor, Eur. J. Pharmacol. 1972, 18, 95) in male CD1 mice of 20-25 g body weight after 16 hours deprivation of food. 30 min before treatment mice were individually placed into small, transparent acrylic cages for habituation. Test substances were administered orally in a volume of 0.1 ml/10 g. 30 min later mice were treated with apomorphine HCl (SIGMA) in a subcutaneous dose of 2 mg/kg. Stereotyped behaviour was observed in every 5th min for 60 min and scored from 0-5. The scores were summed individually, meaned and compared to the control group. The ED₅₀ values were calculated by Litchfield-Wilcoxon's method (J. Pharmacol. Exp. Ther. 1949, 96, 49,).

Results are summarized in Table 6.

TABLE 6
Inhibition of apomorphine-induced climbing
and stereotyped behavior in mice

| Compounds | | |
|---|---|---|
| (Referents and | Climbing | Stereotypy |
| compounds of | ED₅₀ (mg/kg | ED₅₀ (mg/kg |
| Example) | ip.) | po.) |
| Chlorpromazine | 1.2 | 3.6 |
| Clozapine | 2.64 | 22.9 |
| 121 | 4.31 | 3.54 |
| 123 | 9.2 | 7.52 |
| 104 | 7.7 | 6.97 |
| 110 | 14.6 | 3.5 |
| 112 | 25 | Not available |
| 183 | 8.7 | Not available |

As the data of the table show, clozapine was more active in climbing than in the stereotypy assay while the test compounds of the invention inhibited both behaviors, similarly to chlorpromazine.

In order to investigate the potential side effect profile, the catalepsy test in rats has been carried out according to Costall and Naylor (Costall, B., Naylor, R. J. Arzneimittelforschung/Drug Res., 1973, 23, 674).

Catalepsy is defined as a failure to correct an externally imposed, unusual posture over a prolonged time. Neuroleptics which have direct inhibitory action on the nigrostriatal dopamine system induce catalepsy. It may be reflected by the Parkinson-like extrapyramidal symptoms seen clinically with administration of classical antipsychotics. The experiments were carried out in male CDBR rats weighing 300-400 g. The volume of administration was 0.25 ml/100 g body weight. The animals were starved for 16 hours before treatment, water was delivered ad libitum. After intraperitoneal administration of the test substances, the forepaws of the rats were placed on a horizontal stainless steel bar elevated to 10 cm high, while the hind paws remained on a metal plate. The semi-automatic 5-channel catalepsy meter measured the time spent in this unusual posture by an electronic stop-clock. The catalepsy time was scored from 0 to 5 according to the time spent in the unusual posture. The scores were observed in every 30 min for 4 hours and the total scores of 8 readings were summed up individually. Means of groups were calculated.

TABLE 7
Cataleptogenic activity in rats

| Compounds | |
|---|---|
| (Referents and | |
| compounds of | Dose (mg/kg ip)/ |
| Example) | Catalapsy scores |
| Chlorpromazine | 10:8.7 |
| Clozapine | 10:0.14 |
| 121 | 10:0 |
| 123 | 40:0 |
| 104 | 40:0 |
| 110 | 10:0.375 |

Data of Table 7 show that compounds of the invention lack cataleptogenic potential similar to the atypical antipsychotic clozapine while the classical chlorpromazine induces severe catalepsy.

For measuring the antipsychotic potential in a non-perturbed dopamine system the pole jumping assay was used (Cook, L., Catania, A. C. Fed. Proc. 1964, 23, 818.)

This method is an active avoidance learning test. Long-Evans rats, weighing 250-450 g, were used in the experiments. Rats are placed in a box (25×25×25 cm) on a grid floor in the center of which there is a pole. After a latency period, light is turned on for a given time then the floor is electrified to deliver an unpleasant footshock. Rats may learn to avoid the shock by jumping up to grasp the pole. As soon as the rat jumps up the light and shock are turned off. Each light-on period lasted for 15 s, shock time for 30 s with an intertrial interval of 15 s. 30 such trials per day were repeated during the learning period until animals reach a minimum of 80% avoidance rate. During the experimental sessions 20 trials/day were run. Test substances were administered ip. in a volume of 0.25 ml/100 g body weight. Significances were calculated by Student's t test.

During the experiments even those rats which did not jump onto the pole during the light-on period, they jumped during the footshock phase excluding influence on motor function: antipsychotics suppress only the avoidance procedure and not the escape responding (See Table 8).

TABLE 8
Effect on conditioned avoidance responses:
pole jumping test in rats

| Compounds | |
|---|---|
| (Referent and compounds | |
| of Example) | MED (mg/kg ip) |
| Clozapine | 2 |
| 121 | 5 |
| 123 | 2 |
| 104 | 15 |
| 110 | 2 |

MED = minimal effective dose (stat. sign.)

According to the data, some representatives of the compounds of the invention show similar activity to clozapine.

Alterations in the mesocortical dopaminergic tract are thought to be responsible for the negative symptoms of schizophrenia (flattening of affect, poverty of speech, lack of volition and drive, loss of feeling, social withdrawal and decreased spontaneous movement). The best animal model of these symptoms is the behavioral changes induced by PCP (phencyclidine). The PCP-induced stereotypy and hypermotility was investigated in mice according to Ljungberg and Ungerstedt (Ljungberg, T., Ungerstedt, U. Pharmacol. Biochem. Behav., 1985, 23, 479,) and Jawitt (Jawitt, D. C. Amer. J. Psychiatry, 1991, 145, 1301). The stereotypy experiments were carried out in male CD1 mice weighing 20-25 g. The animals were starved for 16 hours before treatment without limitation of water availability. In the stereotypy studies, 30 min before treatment, mice were individually placed into small, transparent acrylic cages for habituation. Test substances were administered orally in a volume of 0.1 ml/10 g. 30 min later mice were treated with PCP with an intraperitoneal dose of 7 mg/kg. Stereotyped behavior was observed every 5th min for 60 min and scored from 0 to 4. The scores were summed individually, averaged and compared to the control group.

The motor activity was measured in a 4-channel activity meter. The apparatus consisted of acrylic cages (40×40×32 cm) equipped with 16 pairs of infrared photocells. The photocells' beam, when broken, signaled a count, which was then recorded by a computer. A 5 mg/kg intraperitoneal dose of PCP was administered 5 min before the experiment. This dose of PCP induces 110-120% increase of the spontaneous motor activity. Test substances were administered orally 15 min before experiment, and mice were investigated individually in the experimental cages for 60 min. 10 mice/group were used. The total counts for each experimental group were compared to the vehicle treated control group.

In both studies the ED₅₀ values were calculated by the Litchfield-Wilcoxon's method (J. Pharmacol. Exp. Ther. 1949, 96, 49).

TABLE 9
Effect in the PCP-induced stereotypy model in mice

| | | |
|---|---|---|
| Compounds | | |
| (Reference and | PCP- | PCP- |
| compounds of | stereotypy | hypermotility |

| | |
|---|---|
| Example) | ED₅₀ mg/kg po. |

| | | |
|---|---|---|
| Chlorpromazine | N.D. | 1.86 |
| Clozapine | 3.76 | 1.81 |
| 121 | 1.56 | 0.87 |
| 123 | 1.07 | 0.62 |
| 104 | 1.12 | 0.73 |
| 110 | 2.38 | 1.4 |

N.D. = not determined

As the data of Table 9 show, selected compounds show a stronger inhibitory activity on PCP-induced behavioral changes compared either to chlorpromazine or clozapine indicating a better chance for influencing the negative symptoms of schizophrenia, too. The results further indicate that the compounds of the invention are active orally.

The antipsychotic activity of compounds 121 and 183 were evaluated using PCP-induced disruption of pre-pulsed inhibition (“PPI”) in C57B1/6J mice. In general, it was found that Clozapine as well as Compound 121 (2.5 and 5 mg/kg) and Compound 183 (5, 10, and 15 mg/kg) significantly reversed phenylcyclohexylpiperidine (PCP)-induced disruption of PPI.

Male C57B1/6J mice from Jackson Laboratories (Bar Harbor, Me.) were used in this study. Mice were received at 6-weeks of age. Upon receipt, mice were assigned unique identification numbers (tail marked) and were group housed in OPTImice cages. All animals remained housed in groups of four during the remainder of the study. All mice were acclimated to the colony room for at least two weeks prior to testing and were subsequently tested at an average age of 8 weeks of age. During the period of acclimation, mice were examined on a regular basis, handled, and weighed to assure adequate health and suitability. Mice were maintained on a 12/12 light/dark cycle with the light on at 6:00 a.m. The room temperature was maintained between 20 and 23° C. with a relative humidity maintained between 30% and 70%. Food and water were provided ad libitum for the duration of the study. In each test, animals were randomly assigned across treatment groups.

All compounds were injected i.p. at a dose volume of 10 ml/kg. Compound 121 (0.5, 2.5, 5 mg/kg) and Compound 183 (1.5, 5, 10, 15 mg/kg) were dissolved in 3% polysorbate tween 80 and sterile isotonic saline. Clozapine (1 mg/kg) was dissolved in 10% DMSO. Phenylcyclohexylpiperidine (PCP) (8 mg/kg) was dissolved in sterile water.

The acoustic startle measured an unconditioned reflex response to external auditory stimulation. PPI consisting of an inhibited startle response (reduction in amplitude) to an auditory stimulation following the presentation of a weak auditory stimulus or prepulse, has been used as a tool for the assessment of deficiencies in sensory-motor gating, such as those seen in schizophrenia. Mice were placed in the PPI chambers (Med Associates) for a 5 min session of white noise (70 dB) habituation.

After the acclimation period the test session was automatically started. The session started with a habituation block of 6 presentations of the startle stimulus alone, followed by 10 PPI blocks of 6 different types of trials. Trial types are: null (no stimuli), startle (120 dB), startle plus prepulse (4, 8 and 12 dB over background noise i.e. 74, 78 or 82 dB) and prepulse alone (82 dB). Trial types were presented at random within each block.

Each trial started with a 50 ms null period during which baseline movements were recorded. There was a subsequent 20 ms period during which prepulse stimuli were presented and responses to the prepulse measured. After further 100 ms the startle stimuli were presented for 40 ms and responses recorded for 100 ms from startle onset. Responses were sampled every ms. The inter-trial interval was variable with an average of 15 s (range from 10 to 20 s). In startle alone trials the basic auditory startle was measured and in prepulse plus startle trials the amount of inhibition of the normal startle was determined and expressed as a percentage of the basic startle response (from startle alone trials), excluding the startle response of the first habituation block.

For PCP-disrupted PPI, mice were pretreated with Vehicle, Compound 121, Compound 183, or Clozapine and placed in holding cages for 30 min following which mice were injected with either PCP or water and placed back in holding cages for 30 min prior to testing.

Data were analyzed by analysis of variance (ANOVA) followed by post-hoc comparisons with Fisher Tests when appropriate. An effect was considered significant if p<0.05. Data are represented as the mean and standard error to the mean. Mice that showed mean startle less than 100 or a response that was 2 standard deviations above or below the mean were removed from the final analysis.

The high doses of both Compound 121 (5 mg/kg) and Compound 183 (15 mg/kg) caused sedation and lethargy in the mice.

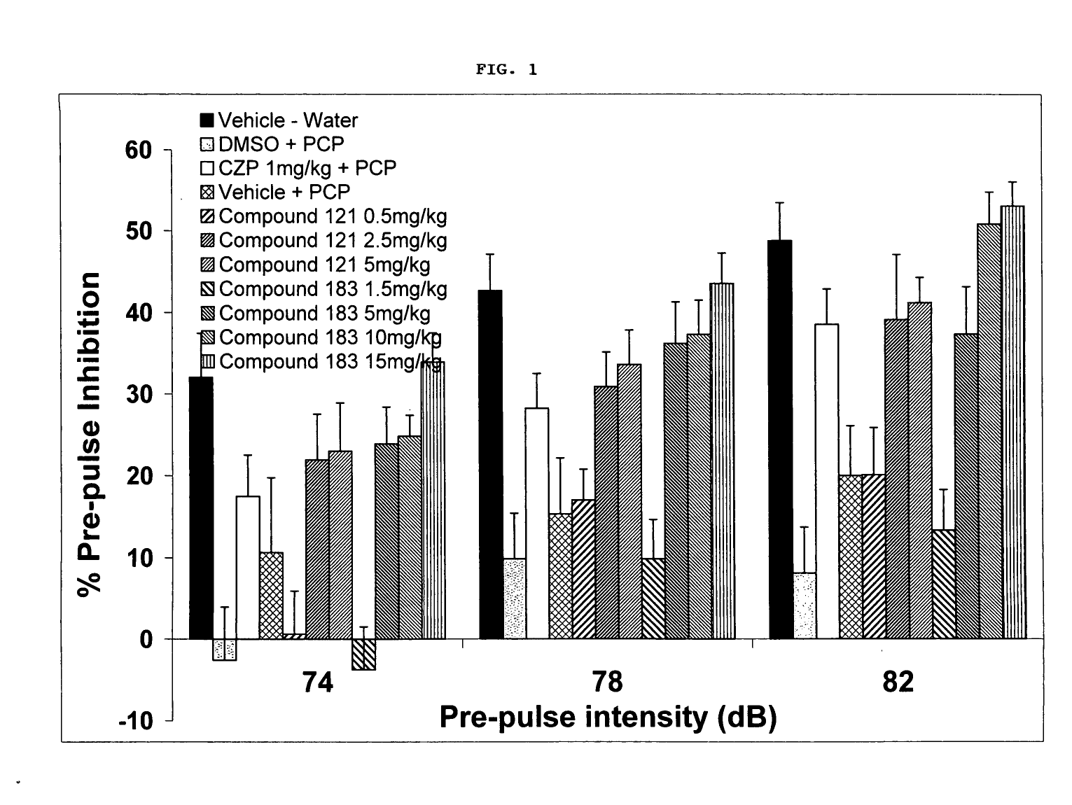

The effects of Compound 121, Compound 183, and Clozapine on PCP-induced disruption of PPI are shown in FIG. 1. Repeated measures ANOVA found a significant treatment effect. Compared to the vehicle alone, PCP significantly disrupted PPI. Pretreatment with Clozapine, Compound 121 (2.5 and 5 mg/kg) and all doses of Compound 183 significantly reversed this disruption.

The effects of compounds 121 and 183, administered orally, were also evaluated in the PCP induced locomotor activity test. Compound 121 was designated as “Compound A” and Compound 183 was designated as “Compound B” for purposes of this study. In general, the study aimed to test Compounds A and B for potential antipsychotic activity in PCP treated mice.

The drugs used were as follows:

Compounds A & B were dissolved in 3% Tween: Compound A: 1, 3, 6, 10, and 15 mg/kg; Compound B: 3, 7, 14, 28, and 40 mg/kg; Compounds A+B were administered by gavage.

Clozapine (1 mg/kg) was dissolved in 10% DMSO.

PCP (5 mg/kg) was dissolved in sterile injectable water.

The open field (“OF”) test assessed both anxiety and locomotor behavior. The open field chambers are Plexiglas square chambers (27.3×27.3×20.3 cm; Med Associates Inc., St Albans, Vt.) surrounded by infrared photobeams (16×16×16) to measure horizontal and vertical activity. The analysis was configured to divide the open field into a center and periphery zone. Distance traveled was measured from horizontal beam breaks as the mouse moved whereas rearing activity was measured from vertical beam breaks.

Mice were brought to the activity experimental room for at least 1 hr acclimation to the experimental room conditions prior to testing. Eight animals are tested in each run. Mice treated with Vehicle, Compound A and B were placed in holding cages for 30 minutes; then placed in the OF chamber for the 30 minute baseline assessment following which, they were injected with PCP (5 mg/kg) or water and placed back in the OF chambers for a 60-minute session. Animals that were injected with either 10% DMSO or Clozapine were placed in the OF immediately for a 30-min baseline assessment, followed by injection of PCP for the 60 minute session. At the end of each OF test session, the OF chambers were thoroughly cleaned.

Data were analyzed by analysis of variance (ANOVA) followed by post-hoc comparisons with Fisher Tests when appropriate. Data was analyzed to show baseline activity (activity during the 30 min prior to PCP injection) and PCP-induced activity (activity during the 60 min after PCP injection). Statistical outliers that fell above or below 2 standard deviations from the mean were removed from the final analysis. An effect was considered significant if p<0.05. Data are represented as the mean and standard error to the mean.

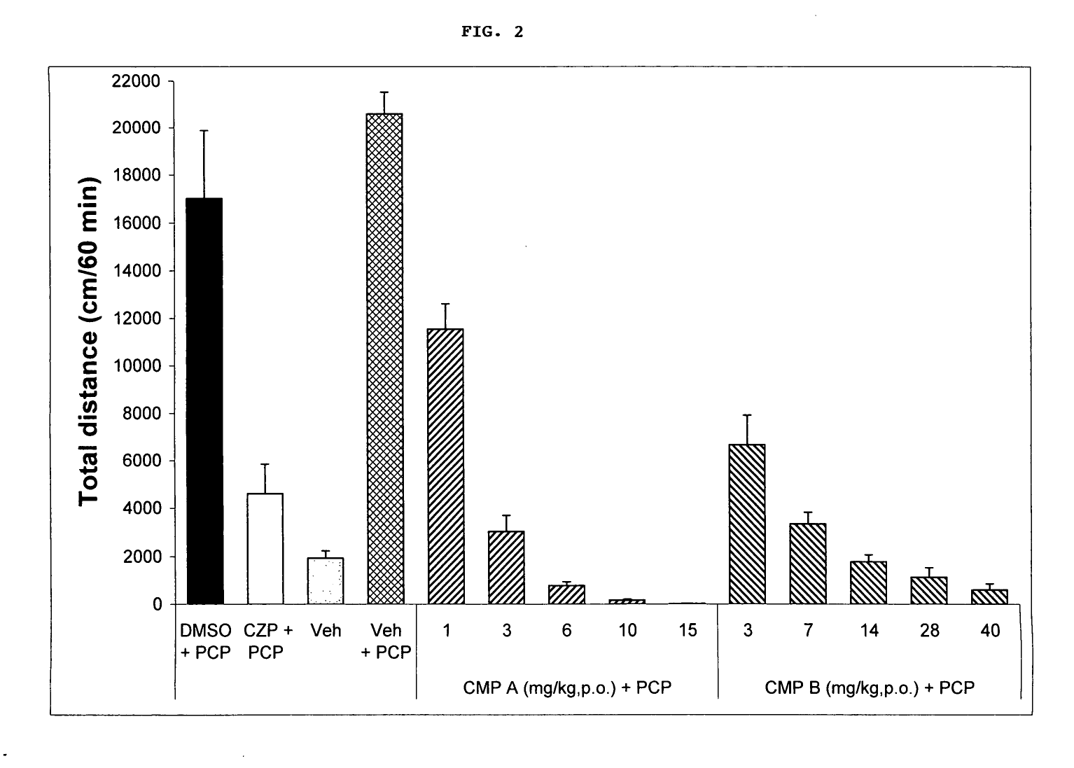

The effect of Compounds A & B on PCP-induced locomotion is shown in FIG. 2. Statistical analysis by ANOVA found a significant treatment effect. Post hoc analysis found that clozapine and all doses of Compounds A & B significantly decreased the PCP induced locomotion, compared to their respective vehicles.

The results of the different pharmacological investigations mentioned above show that the compounds of formula (I) of the invention exert an antipsychotic character and they can be regarded as atypical antipsychotics

1. A compound of formula (I): 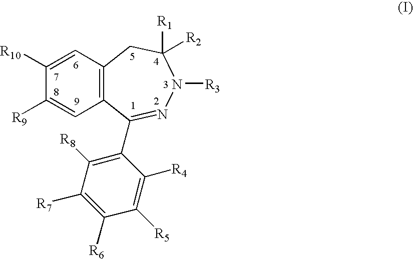
wherein R¹ is methyl and R² is hydrogen;
R³ is:
a) a 5 or 6 membered heterocyclic ring which is either aromatic, saturated or partially saturated, said heterocyclic ring containing 1, 2, or 3 heteroatoms selected from the group consisting of O, S, or N, said heterocyclic ring optionally substituted by a C₁-C₃ alkyl group, a C₂-C₃ alkenyl group or an oxo group, or 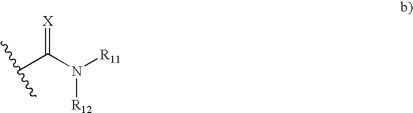
wherein X is O or S;
R¹¹ is hydrogen, C₁-C₄ alkyl or cycloalkyl or phenyl, and
R¹² is C₁-C₄ alkyl, cycloalkyl, phenyl, or C₁-C₃ alkoxy, or
R¹¹ and R¹² together with the nitrogen atom to which they are attached form an imidazolyl or a morpholinyl group, or 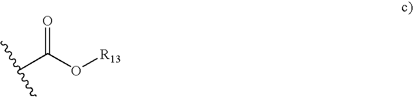
wherein R¹³ is C₁-C₄ alkyl or phenyl;
R⁴, R⁵, R⁶, R⁷, and R⁸ are each independently H, halogen, C₁-C₃ alkyl, nitro, NR¹⁵R¹⁶ wherein R¹⁵ and R¹⁶ can each independently be H, C₁-C₃ alkyl, C₂-C₅ acyl, C₂-C₅ alkoxycarbonyl, aminocarbonyl, or C₁-C₅ alkylaminocarbonyl; and
R⁹ and R¹⁰ are each independently C₁-C₃ alkoxy;
or a stereoisomer or pharmaceutically acceptable salt thereof.

2. The compound of claim 1, wherein R⁶ is nitro.

3. The compound of claim 1, wherein R⁶ is NR¹⁵R¹⁶.

4. The compound of claim 3, wherein R¹⁵ and R¹⁶ are each H.

5. The compound of claim 3, wherein R¹⁵ is H and R¹⁶ is acetyl.

6. The compound of claim 1, wherein R⁴, R⁵, R⁷ and R⁸ are each H.

7. The compound of claim 1, wherein R⁴, R⁷ and R⁸ are each H, and R⁵ is methyl.

8. The compound of claim 1, wherein the stereochemistry of the carbon in the 4-position is in the R-conformation.

9. The compound of claim 1, wherein the stereochemistry of the carbon in the 4-position is in the S-conformation.

10. The compound of claim 1, wherein R⁹ and R¹⁰ are each methoxy.

11. The compound of claim 1, wherein R³ is a 5 or 6 membered heterocyclic ring which is either aromatic, saturated or partially saturated, said heterocyclic ring containing 1, 2, or 3 heteroatoms selected from the group consisting of O, S, or N, said heterocyclic ring optionally substituted by a C₁-C₃ alkyl group, a C₂-C₃ alkenyl group or an oxo group.

12. The compound of claim 11, wherein R³ is a substituted or unsubstituted thiazole, thiazoline, 4-thiazolinone, oxazole, oxazoline, 1,3,4-thiadiazole, 1,3,4-oxadiazole, 1,2,4-thiadiazolin-3-one, 1,2,4-oxadiazole, 4H-1,2,4-oxadiazol-5-one, 1,4,2-oxathiazole, 1,3,4-triazole, pyridine and 5,6-dihydro-4H-[1,3,4]thiadiazin-5-one.

13. The compound of claim 1, wherein R³ is: 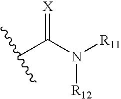
wherein X is O or S;
R¹¹ is hydrogen, C₁-C₄ alkyl or cycloalkyl or phenyl, and R¹² is C₁-C₄ alkyl, cycloalkyl, phenyl, or C₁-C₃ alkoxy, or
R¹¹ and R¹² together with the nitrogen atom to which they are attached form an imidazolyl or a morpholinyl group.

14. The compound of claim 13, wherein X is O.

15. The compound of claim 1, wherein R³ is: 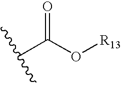
wherein R₁₃ is C₁-C₄ alkyl or phenyl.

16. The compound of claim 11, wherein R³ is 1,3 thiazol-2-yl, R⁹ and R¹⁰ are each methoxy, and the stereochemistry of the carbon in the 4-position is in the R-conformation.

17. The compound of claim 16, wherein the compound is [R]-1-(4-aminophenyl)-7,8-dimethoxy-4-methyl-3-(1,3-thiazol-2-yl)-4,5-dihydro-3H-[2,3]benzodiazepine.

18. The compound of claim 16, wherein the compound is [R]-1-(4-N-acetyl-aminophenyl)-7,8-dimethoxy-4-methyl-3-(1,3-thiazol-2-yl)-4,5-dihydro-3H-[2,3]benzodiazepine.

19. The compound of claim 11, wherein the compound is [R]-1-(4-amino-3-methylphenyl)-7,8-dimethoxy-4-methyl-3-(1,2,4-oxadiazol-3-yl)-4,5-dihydro-3H-[2,3]benzodiazepine.

20. The compound of claim 11, wherein the compound is [R]-1-(4-amino-3-methylphenyl)-7,8-dimethoxy-4-methyl-3-(1,3,4-thiadiazol-2-yl)-4,5-dihydro-3H-[2,3]benzodiazepine.

21. The compound of claim 13, wherein the compound is [R]-1-(4-aminophenyl)-7,8-dimethoxy-4-methyl-3-methylcarbamoyl-4,5-dihydro-3H-[2,3]benzodiazepine.

22. The compound of claim 13, wherein the compound is [R]-1-(4-amino-3-methylphenyl)-7,8-dimethoxy-4-methyl-3-methylcarbamoyl-4,5-dihydro-3H-[2,3]benzodiazepine.

23. A pharmaceutical composition comprising a compound of formula (I) according to claim 1 or a stereoisomer or a pharmaceutically acceptable salt thereof, and a pharmaceutically acceptable carrier.

24. A method for treating psychotic disorders comprising administering to a subject in need thereof a therapeutically effective amount of a compound of claim 1 or a stereoisomer or a pharmaceutically acceptable salt thereof.

25. The method of claim 24, wherein said psychotic disorders are selected from the group consisting of schizophrenia, schizophreniform disorder, schizoaffective disorder, delusional disorder, brief psychotic disorder, shared psychotic disorder, psychotic disorder due to a general medical condition, substance-induced psychotic disorder, psychotic disorder not otherwise specified, bipolar disorder and mood disorders with psychotic symptoms.

26. The method of claim 25, wherein the compound administered is [R]-1-(4-aminophenyl)-7,8-dimethoxy-4-methyl-3-(1,3-thiazol-2-yl)-4,5-dihydro-3H-[2,3]benzodiazepine.

27. The method of claim 25, wherein the compound administered is [R]-1-(4-N-acetyl-aminophenyl)-7,8-dimethoxy-4-methyl-3-(1,3-thiazol-2-yl)-4,5-dihydro-3H-[2,3]benzodiazepine.

28. The method of claim 25, wherein the compound administered is [R]-1-(4-amino-3-methylphenyl)-7,8-dimethoxy-4-methyl-3-(1,2,4-oxadiazol-3-yl)-4,5-dihydro-3H-[2,3]benzodiazepine.

29. The method of claim 25, wherein the compound administered is [R]-1-(4-amino-3-methylphenyl)-7,8-dimethoxy-4-methyl-3-(1,3,4-thiadiazol-2-yl)-4,5-dihydro-3H-[2,3]benzodiazepine.

30. The method of claim 25, wherein the compound administered is [R]-1-(4-aminophenyl)-7,8-dimethoxy-4-methyl-3-methylcarbamoyl-4,5-dihydro-3H-[2,3]benzodiazepine.

31. The method of claim 25, wherein the compound administered is [R]-1-(4-amino-3-methylphenyl)-7,8-dimethoxy-4-methyl-3-methylcarbamoyl-4,5-dihydro-3H-[2,3]benzodiazepine.
